SEQUENCE LISTING (7 sequences)

SEQ ID NO: 1 - DNA, 41 bp, Artificial Sequence
  DNA oligonucleotide
gtaacgatccagctgtcacttggaattctcgggtgccaagg

SEQ ID NO: 2 - DNA, 20 bp, Artificial Sequence
  Second hybridization region
agtgacagctggatcgttac

SEQ ID NO: 3 - DNA, 59 bp, Artificial Sequence
  DNA oligonucleotide
  misc_feature (22)..(27): n is a, c, g, or t
ccttggcacccgagaattccannnnnnaaaaaaaaaaaaaaaaaaaaaaaaaaaaaaaa

SEQ ID NO: 4 - DNA, 20 bp, Artificial Sequence
  First hybridization region
gtaacgatccagctgtcact

SEQ ID NO: 5 - DNA, 21 bp, Artificial Sequence
  First primer region
tggaattctcgggtgccaagg

SEQ ID NO: 6 - DNA, 21 bp, Artificial Sequence
  Second primer region
ccttggcacccgagaattcca

SEQ ID NO: 7 - DNA, 32 bp, Artificial Sequence
  Capture sequence
aaaaaaaaaaaaaaaaaaaaaaaaaaaaaaaa

(19) United States
(12) Patent Application Publication
(10) Pub. No.: US 2022/0275438 A1
(43) Pub. Date: Sep. 1, 2022

(54) LIPID-MODIFIED OLIGONUCLEOTIDES AND METHODS OF USING THE SAME

(71) Applicant: THE REGENTS OF THE UNIVERSITY OF CALIFORNIA, Oakland, CA (US)

(72) Inventors: Zev GARTNER, San Francisco, CA (US); David PATTERSON, San Francisco, CA (US); Eric CHOW, San Francisco, CA (US); Robert WEBER, San Francisco, CA (US); Christopher MCGINNIS, San Francisco, CA (US)

(21) Appl. No.: 17/625,687

(22) PCT Filed: Jul. 8, 2020
(86) PCT No.: PCT/US20/41277

§ 371 Date: Jan. 7, 2022

Related U.S. Application Data
(60) Provisional application No. 62/871,702, filed on Jul. 8, 2019.

(51) Int. Cl. C12Q 1/6869; C12Q 1/686; C12Q 1/6862; C12Q 1/6806

(52) U.S. Cl. CPC C12Q 1/6869; C12Q 1/686; C12Q 1/6862; C12Q 1/6806

(57) ABSTRACT

The disclosure generally relates to methods and applications of single-cell barcoding and methods of nucleotide sequencing using composition comprising lipid-modified oligonucleotides.

### CROSS-REFERENCE TO RELATED APPLICATIONS

This application claims priority to U.S. Provisional Application No. 62/871,702 filed on Jul. 8, 2019, which is incorporated by reference in its entirety.

### TECHNOLOGY FIELD

The disclosure generally relates to methods and applications of single-cell barcoding and methods of nucleotide sequencing using composition comprising lipid-modified oligonucleotides.

### BACKGROUND

Single-cell RNA sequencing has become a powerful tool for mapping transcriptional changes in cells. The main advantage of this technique is the ability to survey a diversity of cells in a sample. All single-cell RNA-sequencing protocols share a common initial step in which transcribed RNA from cells is converted to cDNA. The next step is amplification by methods such as PCR and in vitro transcription (IVT). The subsequent steps, culminating in sequencing, allow the expression level of gene products to be quantified. Isolation and barcoding of RNA from single cells is the first and crucial limiting step in single-cell RNA-seq.

Recently, large scale screens (genetic perturbation using shRNA or CRISPR) combined with single-cell RNA sequencing were performed to understand complex biological phenomena. These have a distinct advantage of easy identification/barcoding of samples by shRNA or CRISPR gRNA sequence. Genetic perturbation techniques can be directly coupled with barcode introduction (i.e., by adding polyA+barcodes to the gRNA or shRNA, themselves), whereas, chemical/drug/patient screens that do not involve genetic manipulation have not been able to be barcoded in a fashion that can be “read-out” via scRNA-seq. See, e.g., Adamson et al., Cell 167(7):1867-82 (2016); Aarts et al., Genes Dev. 31(20):2085-98 (2017); Jaitin et al., Cell 167(7):1883-96 (2016).

In single-cell RNA sequencing assays, multiplexing is traditionally achieved through the addition of molecular barcodes to cDNA fragments or beads, depending the application. This is done after isolating cells either using droplet microfluidics or using microwells. To allow labeling of cDNA from cells isolated in individual droplets (or microwells), beads can be used with real-time PCR (RT) primers that also contain a barcode (or in some cases with the barcode on the bead). RT and barcoding can therefore happen in each individual droplet or well. The current methods have at least the following drawbacks: high cost, low efficiency and low multiplexing capabilities. The current sample multiplexing capacity of commercial droplet microfluidics-based single-cell RNA sequencing is limited to eight due to the number of discrete channels used for cellular emulsion and co-encapsulation with mRNA capture beads.

### SUMMARY OF EMBODIMENTS

The disclosure relates to composition comprising oligonucleotides specifically designed to label cells and compositions of pooled cells labeled with distinct exogenous oligonucleotide barcodes that correspond to different sample preparations (e.g., patients, perturbations, replicates of a single experiment, etc.). By incorporating sample-specific information in the form of lipid-modified exogenous oligonucleotides, sample throughput levels will no longer be limited to being defined by the physical dimensions of microfluidics devices. Enhancing sample multiplexing will reduce the cost of single-cell RNA sequencing, limit technical noise arising from batch effects, and make single-cell transcriptome datasets more informative.

The disclosure relates to compositions and methods of using those compositions for barcoding single cells and for RNA sequencing analysis using lipid-modified oligonucleotides on tissue segments taken from samples of a subject. In some embodiments, the disclosure provides a composition comprising a lipid-conjugated DNA oligonucleotide comprising a lipid moiety, a barcode region, and a capture sequence. In some embodiments, the disclosure provides a composition comprising: (a) a first lipid-conjugated DNA oligonucleotide comprising a lipid moiety and a first primer region; and (b) a second DNA oligonucleotide comprising a second primer region, a barcode region, and a capture sequence, wherein the second primer region is the reverse complement of the first primer region. In some embodiments, the disclosure provides a composition comprising: (a) a first lipid-conjugated DNA oligonucleotide comprising a first lipid moiety, a first hybridization region, and a first primer region; (b) a second lipid-conjugated DNA oligonucleotide comprising a second hybridization region and a second lipid moiety, wherein the second hybridization region is the reverse complement of the first hybridization region; and (c) a third DNA oligonucleotide comprising a second primer region, a barcode region, and a capture sequence, wherein the second primer region is the reverse complement of the first primer region.

In some embodiments, the disclosure provides a method of labeling a cell sample, a method of isolating endogenous DNA from a cell sample, or a method of sequencing nucleic acid sequences from a cell sample, the method comprising: (a) exposing the cell sample to one or a plurality of anchor-lipid modified oligonucleotides disclosed herein or any of the disclosed composition for a time period sufficient for the anchor-lipid modified oligonucleotide to embed itself within a cell membrane of the cell; (b) exposing the cell sample to one or a plurality of labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides; (c) ligating the one or plurality of labeling oligonucleotides to the one or plurality of anchor-lipid modified oligonucleotides; and, optionally (d) detecting the presence of the one or plurality of labeling oligonucleotides by detection of one of more unique nucleotide sequences corresponding to the one or plurality of labeling oligonucleotides; and/or (e) isolating the cell based upon the presence of one or plurality of labeling oligonucleotides, wherein the presence of the one or plurality of labeling oligonucleotides is determined by detection of one of more unique nucleotide sequences corresponding to the one or plurality of labeling oligonucleotides. In some embodiments, the cell sample used in the disclosed methods is a three-dimensional preparation of cells from a tissue sample of a subject with a thickness from about 0.1 microns to about 99 microns. In some embodiments, the cell membrane is a plasma membrane dividing the cytoplasm from a point outside of the cell or wherein the cell membrane is a nuclear membrane.

In some embodiments, the disclosure provides a method of sequencing a nucleic acid from one or a plurality of cells from a sample of a subject comprising: (a) partitioning the one or plurality of cells in a three-dimensional preparation or slice with a thickness from about 0.1 microns to about 99 microns corresponding to a region of the sample into a vessel; (b) labeling the one or plurality of cells corresponding to a region of the sample with an oligonucleotide disclosed herein; (c) isolating the nucleic acid from the one or plurality of cells; and (d) sequencing the nucleic acid from the one or plurality of cells. In some embodiments, the disclosed method further comprises: (e) compiling sequence information from each of the one or plurality of cells. In some embodiments, the disclosed method further comprises correlating the sequence information and/or the expression profile of the nucleic acid to a spatial position of the one or plurality of cells within the sample or within the subject.

In some embodiments, the step of compiling in the disclosed method comprises creating an expression profile of each of the one or plurality of cells corresponding to a region of the sample. In some embodiments, the step of labeling in the disclosed method comprises: (w) exposing the one or plurality of cells to one or a plurality of anchor-lipid modified oligonucleotides for a time period sufficient for the anchor-lipid modified oligonucleotide to embed itself within a cell membrane of the cell; and/or (x) exposing the one or plurality of cells to one or a plurality of labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides; and/or (y) ligating the one or plurality of labeling oligonucleotides to the one or plurality of anchor-lipid modified oligonucleotides; and, optionally (z) detecting the presence of the one or plurality of labeling oligonucleotides by detection of one or more unique nucleotide sequences corresponding to the one or plurality of labeling oligonucleotides. In some embodiments, the step of isolating the cell in the disclosed method comprises isolating the cell based upon the presence of one or plurality of labeling oligonucleotides, wherein the presence of the one or plurality of labeling oligonucleotides is determined by detection of one of more unique nucleotide sequences corresponding to the one or plurality of labeling oligonucleotides. In some embodiments, the one or plurality of anchor-lipid modified oligonucleotides used in the disclosed method comprises: (a) a first lipid-conjugated DNA oligonucleotide comprising a first lipid moiety, a first hybridization region, and a first primer region; (b) a second lipid-conjugated DNA oligonucleotide comprising a second hybridization region and a second lipid moiety, wherein the second hybridization region is the reverse complement of the first hybridization region; and (c) a third DNA oligonucleotide comprising from a second primer region, a barcode region, and a capture sequence, wherein the second primer region is the reverse complement of the first primer region.

In some embodiments, the disclosure provides a method of identifying a spatial position of a pattern of nucleic acid expression within a sample or tissue of a subject, the method comprising: (a) partitioning one or a plurality of cells from the sample or the tissue corresponding to a region of the sample into one of a plurality of vessels; (b) exposing the one or plurality of cells corresponding to a region of the sample with an known oligonucleotide disclosed herein for a time sufficient for incorporation of the known oligonucleotide into the one or plurality of cells, each oligonucleotide unique for and corresponding to one of the plurality of vessels into which one or plurality of cells are exposed; (c) isolating nucleic acid from the one or a plurality of cells according to the known oligonucleotide; (d) quantifying expression of nucleic acids and/or sequencing the nucleic acid from the one or a plurality of cells; (e) normalizing the expression of nucleic acid in an expression profile; and (f) correlating the expression profile from the one or plurality of cells to the spatial position of the cells within the sample, relative to the tissue, or within the tissue, wherein the known oligonucleotide disclosed herein in each of the plurality of vessels is independently selected from one or a combination of: (x) a first lipid-conjugated DNA oligonucleotide comprising a first lipid moiety, a first hybridization region, and a first primer region; and/or (y) a second lipid-conjugated DNA oligonucleotide comprising a second hybridization region and a second lipid moiety, wherein the second hybridization region is the reverse complement of the first hybridization region; and/or (z) a third DNA oligonucleotide comprising from a second primer region, a barcode region, and a capture sequence, wherein the second primer region is the reverse complement of the first primer region; wherein the step of partitioning comprises placing a slice or three-dimensional preparation of the sample from about 0.1 to about 99 microns in thickness into one of a plurality of vessels. In some embodiments, the disclosed method further comprises: (g) exposing the cells to flow cytometry. In some embodiments, the step of isolating the cells in the disclosed method comprises exposing the cells to flow cytometry.

In some embodiments, the disclosure provides a method of sequencing a nucleic acid from one or a plurality of cells from a sample of a subject comprising: (a) partitioning the one or plurality of cells corresponding to a region of the sample into a vessel; (b) labeling the one or plurality of cells corresponding to a region of the sample with an oligonucleotide disclosed herein; (c) isolating the nucleic acid from the one or a plurality of cells; and (d) sequencing the nucleic acid from the one or a plurality of cells, wherein the step of partitioning comprises placing a slice or three-dimensional preparation of the sample from about 0.1 to about 99 microns in thickness into one of a plurality of vessels. In some embodiments, the disclosed method further comprises (e) compiling sequence information from each of the one or plurality of cells. In some embodiments, the disclosed method further comprises correlating the sequence information and/or the expression profiles of the nucleic acid to a spatial position of the one or plurality of cells within the sample or within the subject.

In some embodiments, the step of compiling of the disclosed method comprises creating an expression profile of each of the one or plurality of cells corresponding to a region of the sample. In some embodiments, the step of labeling of the disclosed method comprises: (w) exposing the one or plurality of cells to one or a plurality of anchor-lipid modified oligonucleotides for a time period sufficient for the anchor-lipid modified oligonucleotide to embed itself within a cell membrane of the cell; and/or (x) exposing the one or plurality of cells to one or a plurality of labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides; and/or (y) ligating the one or plurality of labeling oligonucleotides to the one or plurality of anchor-lipid modified oligonucleotides; and, optionally (z) detecting the presence of the one or plurality of labeling oligonucleotides by detection of one of more unique nucleotide sequences corresponding to the one or plurality of labeling oligonucleotides. In some embodiments, the step of isolating the nucleic acid of the one or plurality of cells of the disclosed method comprises isolating the one or plurality of cells based upon the presence of one or plurality of labeling oligonucleotides, wherein the presence of the one or plurality of labeling oligonucleotides is determined by detection of one of more unique nucleotide sequences corresponding to the one or plurality of labeling oligonucleotides.

In some embodiments, the disclosure provides a method of identifying a spatial expression pattern of a nucleic acid within a tissue of a subject, the method comprising: (a) partitioning one or a plurality of cells from a sample corresponding to a region of the tissue into one of a plurality of vessels; (b) exposing the one or plurality of cells with a lipid-conjugated DNA oligonucleotide comprising a lipid moiety, a barcode region, and a capture sequence for a time period sufficient for the lipid moiety to embed itself within cell membrane of the one or plurality of cells, wherein the barcode region of the lipid-conjugated DNA oligonucleotide is unique for each of the one of plurality of vessels in which the one or plurality of cells are exposed; (c) sequencing nucleic acids captured by the capture sequence of the lipid-conjugated DNA oligonucleotide in the one or plurality of cells; and (d) correlating the sequenced nucleic acids from the one or plurality of cells to the spatial position of the one or plurality of cells within the tissue and/or relative to the tissue according to the barcode region contained in each of the sequenced nucleic acids. In some embodiments, the lipid-conjugated DNA oligonucleotide used in the disclosed method comprises: (i) a first lipid-conjugated DNA oligonucleotide comprising the lipid moiety and a first primer region; and (ii) a second DNA oligonucleotide comprising a second primer region, the barcode region, and the capture sequence, wherein the second primer region is the reverse complement of the first primer region. In some embodiments, the first lipid-conjugated DNA oligonucleotide further comprises a first hybridization region. In some embodiments, the disclosed method further comprises exposing the one or plurality of cells to a second lipid-conjugated DNA oligonucleotide before the step of sequencing, wherein the second lipid-conjugated DNA oligonucleotide comprises a second hybridization region and a second lipid moiety, wherein the second hybridization region is the reverse complement of the first hybridization region. In some embodiments, the disclosed method further comprises (e) exposing the cells to flow cytometry. In some embodiments, the step of partitioning of the disclosed method comprises placing a tissue slice or a three-dimensional preparation of the sample from about 0.1 to about 99 microns in thickness into the one of plurality of vessels. In some embodiments, the one or plurality of vessels used in the disclosed method are multiwells. In some embodiments, the sample used in the disclosed method is a tissue slice.

In some embodiments, the first lipid-conjugated DNA oligonucleotide of the disclosure, including the disclosed compositions and methods, comprises, from a 5′ to 3′ orientation, the first lipid moiety, the first hybridization region, and the first primer region. In other embodiments, the first lipid-conjugated DNA oligonucleotide of the disclosure, including the disclosed compositions and methods, comprises, from a 3′ to 5′ orientation, the first lipid moiety, the first hybridization region, and the first primer region.

In some embodiments, the second lipid-conjugated DNA oligonucleotide of the disclosure, including the disclosed compositions and methods, comprises, from a 5′ to 3′ orientation, the second hybridization region and the second lipid moiety. In some embodiments, the second lipid-conjugated DNA oligonucleotide of the disclosure, including the disclosed compositions and methods, comprises, from a 3′ to 5′ orientation, the second hybridization region and the second lipid moiety.

In some embodiments, the third DNA oligonucleotide of the disclosure, including the disclosed compositions and methods, comprises, from a 5′ to 3′ orientation, the second primer region, the barcode region, and the capture sequence. In some embodiments, the third DNA oligonucleotide of the disclosure, including the disclosed compositions and methods, comprises, from a 3′ to 5′ orientation, the second primer region, the barcode region, and the capture sequence.

In some embodiments, the first lipid moiety used in the first lipid-conjugated DNA oligonucleotide of the disclosure, including the disclosed compositions and methods, comprises a fatty acid having from about 12 to about 28 carbons. In some embodiments, the second lipid moiety used in the second lipid-conjugated DNA oligonucleotide of the disclosure, including the disclosed compositions and methods, comprises a fatty acid having from about 12 to about 28 carbons. In some embodiments, both the first lipid moiety and the second lipid moiety used in the lipid-conjugated DNA oligonucleotides of the disclosure, including the disclosed compositions and methods, comprise a fatty acid having from about 12 to about 28 carbons.

In some embodiments, the first lipid moiety used in the first lipid-conjugated DNA oligonucleotide of the disclosure, including the disclosed compositions and methods, comprises a compound of Formula I:

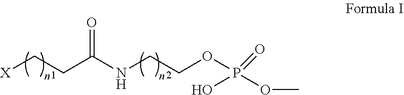

or a physiologically acceptable salt thereof,
wherein n¹ is from 5 to 25, n² is from 1 to 25, and X is selected from the group consisting of NH, CH₂, O, and CH—R, wherein R is a C12 to C28 monoglyceride, alkenyl, alkyl, aryl, or aralkyl. In some embodiments, the first lipid moiety comprises a lipid selected from lignoceric acid and cholesterol. In some embodiments, the cholesterol is cholesterol-triethylene glycol (TEG).

In some embodiments, the second lipid moiety used in the second lipid-conjugated DNA oligonucleotide of the disclosure, including the disclosed compositions and methods, comprises a compound of Formula II:

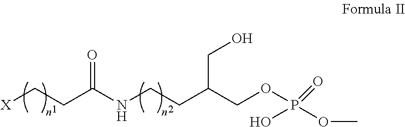

or a physiologically acceptable salt thereof,
wherein n¹ is from 5 to 25, n² is from 0 to 24, and X is selected from the group consisting of NH, CH₂, O, and CH—R, wherein R is a C12 to C28 monoglyceride, alkenyl, alkyl, aryl, or aralkyl. In some embodiments, the second lipid moiety comprises a lipid selected from palmitic acid and cholesterol. In some embodiments, the cholesterol is cholesterol-triethylene glycol (TEG).

In some embodiments, the capture sequence used in the third DNA oligonucleotide of the disclosure, including the disclosed compositions and methods, is a polyadenylation region.

In some embodiments, the first lipid-conjugated DNA oligonucleotide of the disclosure, including the disclosed compositions and methods, comprises a nucleic acid sequence having at least about 70% sequence identity to the nucleic acid sequence of SEQ ID NO: 1 (GTAACGATCCAGCTGTCACTTGGAATTCTCGGGTGCCAAGG). In some embodiments, the second lipid-conjugated DNA oligonucleotide of the disclosure, including the disclosed compositions and methods, comprises a nucleic acid sequence having at least about 70% sequence identity to the nucleic acid sequence of SEQ ID NO: 2 (AGTGACAGCTGGATCGTTAC). In some embodiments, the third DNA oligonucleotide of the disclosure, including the disclosed compositions and methods, comprises a nucleic acid sequence having at least 70% sequence identity to the nucleic acid sequence of SEQ ID NO: 3 (CCTTGGCACCCGAGAATTCCANNNN NNAAAAAAAAAAAAAAAAAAAAAAAAAAAAAAAA).

In some embodiments, the first lipid-conjugated DNA oligonucleotide of the disclosure, including the disclosed compositions and methods, is bound to a solid support. In some embodiments, the second lipid-conjugated DNA oligonucleotide of the disclosure, including the disclosed compositions and methods, is bound to a solid support. In some embodiments, the third lipid-conjugated DNA oligonucleotide of the disclosure, including the disclosed compositions and methods, is bound to a solid support. In some embodiments, one or more of the first lipid-conjugated DNA oligonucleotide, the second lipid-conjugated DNA oligonucleotide, and the third lipid-conjugated DNA oligonucleotide is bound to a solid support. In some embodiments, the solid support is a bead. In some embodiments, the capture sequence used in the third DNA oligonucleotide of the disclosure, including the disclosed compositions and methods, comprises a polyadenylation region and the bead comprises a poly(T) region that hybridizes to the polyadenylation region of the third DNA oligonucleotide.

### BRIEF DESCRIPTION OF DRAWINGS

FIG. 1 shows a schematic and an example of results from the adult gut experiment used as representative examples for the barcode classification workflow. Note the data are taken from samples of tissue that are over 1 cm in thickness and are only an example of the data one could collect in collection with prophetic methods provided in the Examples.

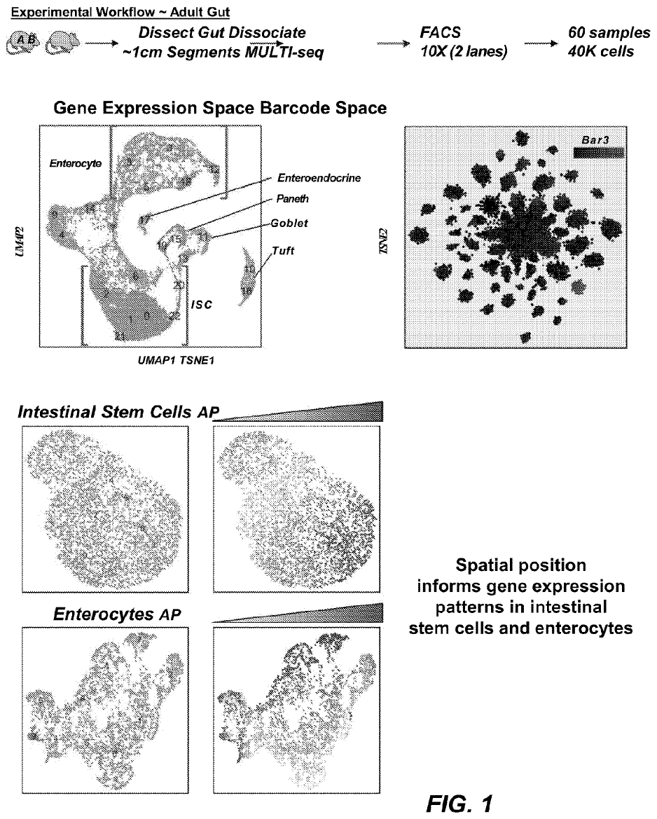

FIG. 2 shows results from the developing gut experiment used as representative examples for the barcode classification workflow. Note the data are taken from samples of tissue that are over 1 cm in thickness and are only an example of the data one could collect in collection with prophetic methods provided in the Examples.

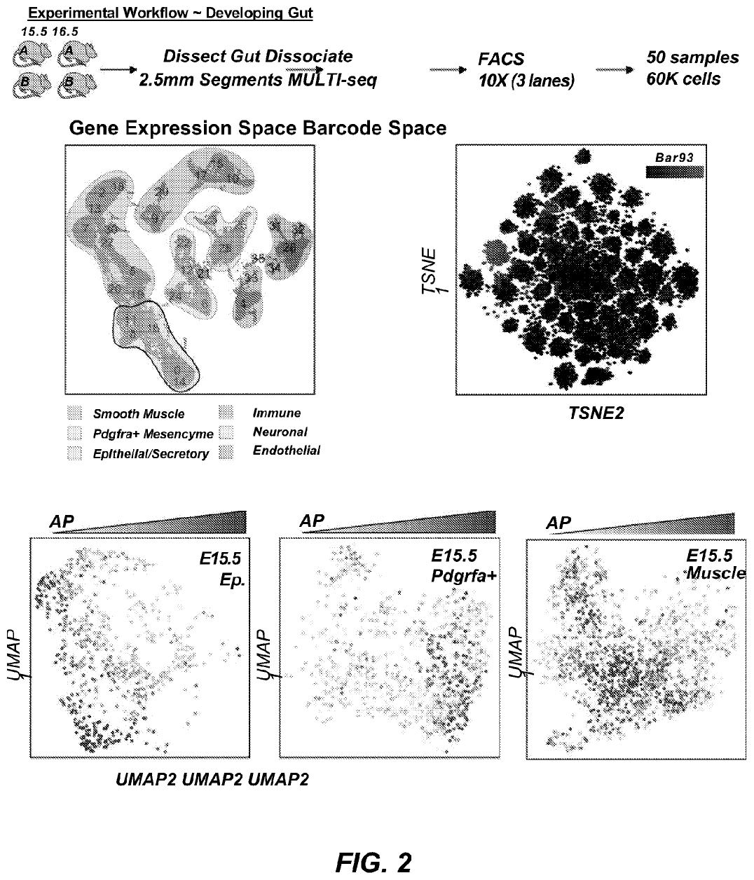

### DETAILED DESCRIPTION OF EMBODIMENTS

Before exemplary embodiments are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither or both limits are included in the smaller ranges is also encompassed within the present disclosure, subject to any specifically excluded limit in the stated range. Where the stated range includes one or both of the limits, ranges excluding either or both of those included limits are also included in the present disclosure.

Unless defined otherwise, all technical and scientific terms used herein have the same meaning as commonly understood by one of ordinary skill in the art to which this present disclosure belongs. Although any methods and materials similar or equivalent to those described herein can be used in the practice or testing of the disclosed embodiments, some potential and exemplary methods and materials may now be described. Any and all publications mentioned herein are incorporated herein by reference in their entireties to disclose and describe the methods and/or materials in connection with which the publications are cited. It is understood that the present disclosure supersedes any disclosure of an incorporated publication to the extent there is a contradiction.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present disclosure is not entitled to antedate such publication. Further, the dates of publication provided may he different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can he carried out in the order of events recited or in any other order which is logically possible.

### Definitions

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims.

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials are now described. All publications mentioned herein are incorporated by reference. Nothing herein is to be construed as an admission that the disclosure is not entitled to antedate such disclosure by virtue of prior disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “endogenous” as used herein refers to a substance that is originating from within an organism. An “endogenous” nucleic acid therefore refers to a nucleic acid that is originated or produced within an organism, tissue or cell.

As used herein, the term “normalizing” or “normalized” refers to an expression level of a nucleic acid relative to the mean expression levels of one or a set of a reference nucleic acids. The reference nucleic acids are based on their minimal variation across tissues or cells.

The term “labeling oligonucleotide” refers to an oligonucleotide comprising a sequence that is complimentary to at least one portion of the anchor-lipid modified oligonucleotide defined herein so that the anchor-lipid modified oligonucleotide form a complementary strand of nucleic acid with the labeling oligonucleotide. In some embodiments, the labeling oligonucleotide comprises a primer region as defined herein. In some embodiments, the labeling oligonucleotide comprises a barcode region as defined elsewhere herein. In some embodiments, the labeling oligonucleotide comprises a capture sequence as defined herein.

The term “lipid-modified oligonucleotide”, “lipid-DNA”, “hydrophobic-anchored oligonucleotide” and similar terms are to be broadly construed to include any oligonucleotide or polynucleotide that is attached by any means to a hydrophobic, lipophilic, or amphiphilic region that can be inserted into a membrane, regardless of whether the “lipid-modified oligonucleotide”, “lipid-DNA”, “hydrophobic-anchored oligonucleotide”, or portion thereof is actually inserted into a membrane.

The term “membrane” or any similar term is used broadly and generically herein to refer to any lipid-containing membrane, cellular membrane, nuclear membrane, monolayer, bilayer, vesicle, liposome, lipid bilayer, etc., and the present disclosure is not meant to be limited to any particular membranes.

As used herein, the term “subject,” “individual” or “patient,” used interchangeably, means any animal, including mammals, such as mice, rats, other rodents, rabbits, dogs, cats, swine, cattle, sheep, horses, or primates, such as humans.

As used herein, the term “kit” refers to a set of components provided in the context of a system for sequencing nucleotides and/or isolating nucleotides sequences and/or diagnosing a subject with having a disease or infection based upon the presence, absence and/or quantity of expressed nucleotide sequences from a sample or a cell. In some embodiments, the term kit refers to a set of components provided in the context of a system for sequencing nucleotides and/or isolating nucleotides sequences and/or diagnosing a subject with having a disease or infection based upon the spatial location of expressed nucleotide sequences in a sample or a cell. Such systems may include, for example, systems that allow for storage, identification, or delivery of expressed genes in one or a plurality of cells (e.g., oligonucleotides, oligonucleotides that encode enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a diagnostic assay comprising two or more separate containers that each contain a subportion of total kit components. Containers may be delivered to an intended recipient together or separately. For example, a first container may contain a solid support or polystyrene plate for use in a cell culture assay, while a second container may contain cells, such as control cells. As another example, the kit may comprise a first container comprising a solid support such as a chip or slide with one or a plurality of ligands with affinities to one or a plurality of biomarkers disclosed herein and a second container comprising any one or plurality of reagents necessary for the detection and/or quantification of the amount of lipid-modified oligonucleotides in a sample. The term “fragmented kit” is intended to encompass kits containing Analyte Specific Reagents (ASR's) regulated under section 520(e) of the Federal Food, Drug, and Cosmetic Act, but are not limited thereto. Any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human. In some embodiments, the mammal refers to any non-human mammal. The present disclosure relates to any of the methods or compositions of matter disclosed herein wherein the sample is taken from a mammal or non-human mammal. The present disclosure relates to any of the methods or compositions of matter disclosed herein wherein the sample is taken from a human.

As used herein, the phrase “in need thereof” means that the animal or mammal has been identified or suspected as having a need for the particular method or treatment that is needed based upon the presence, absence and/or quantity of a biomarker. In some embodiments, the identification can be by any means of diagnosis or observation. In any of the methods and treatments described herein, the animal or mammal can be in need thereof. In some embodiments, the animal or mammal is in an environment or will be traveling to an environment in which a particular disorder or condition is prevalent or more likely to occur.

The particular use of terms “nucleic acid,” “oligonucleotide,” and “polynucleotide” should in no way be considered limiting and may be used interchangeably herein. “Oligonucleotide” is used when the relevant nucleic acid molecules typically comprise less than about 100 bases. “Polynucleotide” is used when the relevant nucleic acid molecules typically comprise more than about 100 bases. Both terms are used to denote DNA, RNA, modified or synthetic DNA or RNA (including, but not limited to nucleic acids comprising synthetic and naturally-occurring base analogs, dideoxy or other sugars, thiols or other non-natural or natural polymer backbones), or other nucleobase containing polymers capable of hybridizing to DNA and/or RNA. Accordingly, the terms should not be construed to define or limit the length of the nucleic acids referred to and used herein, nor should the terms be used to limit the nature of the polymer backbone to which the nucleobases are attached.

Polynucleotides of the present disclosure may be single-stranded, double-stranded, triple-stranded, or include a combination of these conformations. Generally polynucleotides contain phosphodiester bonds, although in some cases, as outlined below, nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include morpholinos, locked nucleic acids (LNAs), as well as those with positive backbones, non-ionic backbones, and non-ribose backbones. Nucleic acids containing one or more carbocyclic sugars are also included within the definition of nucleic acids. These modifications of the ribose-phosphate backbone may be done to facilitate the addition of additional moieties such as labels, or to increase the stability and half-life of such molecules in physiological environments.

The term “nucleic acid sequence” or “polynucleotide sequence” refers to a contiguous string of nucleotide bases and in particular contexts also refers to the particular placement of nucleotide bases in relation to each other as they appear in a polynucleotide.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “fluorogenic probe” refers to any molecule (dye, peptide, or fluorescent marker) that emits a known and/or detectable wavelength of light upon exposure to a known wavelength of light. In some embodiments, the substrates or peptides with known cleavage sites recognizable by any of the enzymes expressed by the one or plurality of animals or single-cell organisms. In some embodiments, the fluorgenic probe is attached to a any of the one or plurality of oligonucleotide sequences disclosed herein. In some embodiments, the attachment of the fluorogenic probe to the oligonucleotides disclosed herein creates a chimeric molecule capable of a fluorescent emission or emissions upon exposure of the substrate to the enzyme and the known wavelength of light, such that exposure to an enzyme creates a reaction product which is quantifiable in the presence of a fluorimeter or spectrophotometer. In some embodiments, the fluorogenic probe is fully quenched upon exposure to the known wavelength of light before enzymatic cleavage of the substrate and the fluorogenic probe emits a known wavelength of light the intensity of which is quantifiable by absorbance readings or intensity levels in the presence of a fluorimeter and, optionally, after cleavage of the probe from the oligonucleotide on which is bounde. In some embodiments, the fluorogenic probe is a coumarin-based dye or rhodamine-based dye with fluorescent emission spectra measureable or quantifiable in the presence of or exposure to a predetermined wavelength of light. In some embodiments, the fluorogenic probe comprises rhodamine. In some embodiments, the fluorogenic probe comprises rhodamine-100. Coumarin-based fluorogenic probes are known in the art, for example in a U.S. Pat. Nos. 7,625,758 and 7,863,048, which are herein incorporated by reference in their entireties. In some embodiments, the fluorogenic probes are a component to, covalently bound to, non-covalently bound to, intercalated with one or a plurality of substrates to any of the enzymes disclosed herein. In some embodiments, the fluorogenic probes are chosen from ACC or AMC. In some embodiments, the fluorogenic probe is a fluorescein molecule. In some embodiments, the fluorogenic probe is capable of emitting a resonance wave detectable and/or quantifiable by a fluorimeter after exposure to one or a plurality of enzymes catalyzing the cleavage of one or a plurality of lipid-modified oligonucleotides disclosed herein.

As used herein, the term “score” refers to a single value or range of values that can be used as a component in a predictive model for the diagnosis, prognosis, or clinical treatment plan for a subject, wherein the single value is calculated by combining and/or normalizing raw data values with or against a control value based upon features or metrics measured in the system. In some embodiments, the score is calculated by through an interpretation function or algorithm. In some embodiments, the subject is suspected of having expression of a gene that promotes or contributes to the likelihood of acquiring a disease state or whose expression is correlative to the presence of a pathogen. Calculation of score can be accomplished using known algorithms executable in computer program products within equipment used in sequencing or analyzing samples. For instance, in the case of using a BioRAD ddSEQ Single-Cell Isolator, algorithms required to detect, quantify or visualize probes and/or barcodes labeled with probes are described in BioRad's Publication 7139, entitled “Implementing the Drop-Seq Protocol on Bio-Rad's ddSEQ Single-Cell Isolator” found at https://www.bio-rad.com/en-us/product/ddseq-single-cell-isolator?ID=OKNWBSE8Z, the contents of which are incorporated by reference in its entirety. In some embodiments, the methods disclosed herein comprise substeps of detecting the presence absence or quantity of a given barcode oligonucleotide by calculating the quantity of a probe in a control sample, calculating the quantity of a probe in the cell sample and normalizing the signal obtained from the cell sample by subtracting the signal obtained from the control sample.

To facilitate the detection of a lipid-modified oligonucleotide disclosed herein, such as a detectable substance may be pre-applied to a surface, for example a plate, well, bead, or other solid support comprising one or a plurality of reaction vessels. In some embodiments, sample may be pre-mixed with a diluent or reagent before it is applied to a surface. The detectable substance may function as a lipid-oligonucleotide that is detectable either visually or by an instrumental device. Any substance generally capable of producing a signal that is detectable visually or by an instrumental device may be used as detection probes. Suitable detectable substances may include, for instance, luminescent compounds (e.g., fluorescent, phosphorescent, etc.); radioactive compounds; visual compounds (e.g., colored dye or metallic substance, such as gold); liposomes or other vesicles containing signal-producing substances; enzymes and/or substrates, and so forth. Other suitable detectable substances may be described in U.S. Pat. No. 5,670,381 to Jou, et al. and U.S. Pat. No. 5,252,459 to Tarcha, et al., which are incorporated herein in their entirety by reference thereto for all purposes. If the detectable substance is colored, the ideal electromagnetic radiation is light of a complementary wavelength. For instance, blue detection probes strongly absorb red light. In some embodiments, the lipid-modified oligonucleotide comprises a probe. In some embodiments, the detectable probe comprises or consists of a luminescent compound that produces an optically detectable signal that corresponds to the level or quantity of lipid-oligonucleotide in the sample. For example, suitable fluorescent molecules may include, but are not limited to, fluorescein, europium chelates, phycobiliprotein, phycoerythrin, phycocyanin, allophycocyanin, o-phthaldehyde, fluorescamine, rhodamine, and their derivatives and analogs. Other suitable fluorescent compounds are semiconductor nanocrystals commonly referred to as “quantum dots.” For example, such nanocrystals may contain a core of the formula CdX, wherein X is Se, Te, S, and so forth. The nanocrystals may also be passivated with an overlying shell of the formula YZ, wherein Y is Cd or Zn, and Z is S or Se. Other examples of suitable semiconductor nanocrystals may also be described in U.S. Pat. No. 6,261,779 to Barbera-Guillem, et al. and U.S. Pat. No. 6,585,939 to Dapprich, which are incorporated herein in their entirety by reference thereto for all purposes.

Further, suitable phosphorescent compounds may include metal complexes of one or more metals, such as ruthenium, osmium, rhenium, iridium, rhodium, platinum, indium, palladium, molybdenum, technetium, copper, iron, chromium, tungsten, zinc, and so forth. Especially preferred are ruthenium, rhenium, osmium, platinum, and palladium. The metal complex may contain one or more ligands that facilitate the solubility of the complex in an aqueous or non-aqueous environment. For example, some suitable examples of ligands include, but are not limited to, pyridine; pyrazine; isonicotinamide; imidazole; bipyridine; terpyridine; phenanthroline; dipyridophenazine; porphyrin; porphine; and derivatives thereof. Such ligands may be, for instance, substituted with alkyl, substituted alkyl, aryl, substituted aryl, aralkyl, substituted aralkyl, carboxylate, carboxaldehyde, carboxamide, cyano, amino, hydroxy, imino, hydroxycarbonyl, aminocarbonyl, amidine, guanidinium, ureide, sulfur-containing groups, phosphorus containing groups, and the carboxylate ester of N-hydroxy-succinimide.

Porphyrins and porphine metal complexes possess pyrrole groups coupled together with methylene bridges to form cyclic structures with metal chelating inner cavities. Many of these molecules exhibit strong phosphorescence properties at room temperature in suitable solvents (e.g., water) and an oxygen-free environment. Some suitable porphyrin complexes that are capable of exhibiting phosphorescent properties include, but are not limited to, platinum (II) coproporphyrin-I and III, palladium (II) coproporphyrin, ruthenium coproporphyrin, zinc(II)-coproporphyrin-I, derivatives thereof, and so forth. Similarly, some suitable porphine complexes that are capable of exhibiting phosphorescent properties include, but not limited to, platinum(II) tetra-meso-fluorophenylporphine and palladium(II) tetra-meso-fluorophenylporphine. Still other suitable porphyrin and/or porphine complexes are described in U.S. Pat. No. 4,614,723 to Schmidt, et al.; U.S. Pat. No. 5,464,741 to Hendrix; U.S. Pat. No. 5,518,883 to Soini; U.S. Pat. No. 5,922,537 to Ewart. et al.; U.S. Pat. No. 6,004,530 to Sagner, et al.; and U.S. Pat. No. 6,582,930 to Ponomarev, et al., which are incorporated herein in their entirety by reference thereto for all purposes.

As used herein, “sequence identity” is determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). To use the term “homologus to” is synonymous with a measured “sequence identity.”

As used herein, the term “sample” refers generally to a limited quantity of something which is intended to be similar to and represent a larger amount of that thing. In the present disclosure, a sample is a collection, swab, brushing, scraping, biopsy, removed tissue, or surgical resection that is to be tested for an assay or method disclosed herein. In some embodiments, samples are taken from a patient or subject that is believed to comprise a hyperproliferative cell. In some embodiments, a sample believed to contain an infection is compared to a “control sample” that is known not to contain one or plurality of cells. In some embodiments, a sample believed to contain a pathogen cell is compared to a control sample that is known to not contain a pathogen cell. In some embodiments, a sample believed to contain a hyperproliferative cell is compared to a control sample that is known not to contain a hyperproliferative cell. In some embodiments, the sample is a brushing of an environmental are or location, such as a lab bench or medical device. This disclosure contemplates using any one or a plurality of disclosed methods herein to identify, detect, and/or quantify the amount of potentially harmful expression of genes or the amount of harmful pathogens or harmful cells on a particular item or location based upon the expression of harmful genes or nucleotide sequences.

The terms “complementary” or “complementarity” refer to polynucleotides (i.e., a sequence of nucleotides) related by base-pairing rules, for example, the sequence “5′-AGT-3′,” is complementary to the sequence “5′-ACT-3′”. Complementarity may be “partial,” in which only some of the nucleic acids' bases are matched according to the base pairing rules, or there may be “complete” or “total” complementarity between the nucleic acids. The degree of complementarity between nucleic acid strands can have significant effects on the efficiency and strength of hybridization between nucleic acid strands under defined conditions. This is of particular importance for methods that depend upon binding between nucleic acid bases.

Any probe disclosed herein may be an antibody. The term “antibody” as used herein refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. As used herein, a “targeted binding agent” is an antibody, or binding fragment thereof, that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope or antigen on the surface of a cell comprising one or more of the modified oligonucleotides disclosed herein. “Binding fragments” of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay). An antibody may be oligoclonal, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known See, for example, Bowie et al. Science 253:164 (1991), which is incorporated by reference in its entirety. Antibodies can be fragmented using conventional techniques and the fragments screened for utility in the same manner as described above for the whole antibodies. For example, F(ab′)2 fragments can be generated by treating antibody with pepsin. The resulting F(ab′)2 fragment can be treated to reduce disulfide bridges to produce Fab′ fragments.

The disclosure also contemplates using one or a plurality of chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes the selected antigens on the surface of differentiated cells or tumor cells. See, for example, Morrison et al., 1985; Proc. Natl. Acad. Sci. U.S.A. 81, 6851; Takeda et al., 1985, Nature 314:452; Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B. In any of the disclosed methods, the methods may comprise exposing any antibody that have an affinity for any of the reaction products created by cleavage of a known substrate after exposure of the substrate to any one or plurality of enzymes set forth in Table 1.

Chemical conjugation is based on the use of homo- and heterobifunctional reagents with E-amino groups or hinge region thiol groups. Homobifunctional reagents such as 5,5′-Dithiobis(2-nitrobenzoic acid) (DNTB) generate disulfide bonds between the two Fabs, and o-phenylenedimaleimide (O-PDM) generate thioether bonds between the two Fabs (Brenner et al., 1985, Glennie et al., 1987). Heterobifunctional reagents such as N-succinimidyl-3-(2-pyridylditio)propionate (SPDP) combine exposed amino groups of antibodies and Fab fragments, regardless of class or isotype (Van Dijk et al., 1989).

Various formats may be used to test for the presence or absence of a lipid-modified oligonucleotide or nucleic acid sequence or functional fragment thereof in a sample or cell isolated from a subject using the assay devices of the present disclosure. For instance, a “sandwich” format typically involves mixing the test sample with lipid-modified nucleic acid sequences conjugated with a specific binding member (e.g., antibody) for the analyte to form complexes between the analyte and the conjugated probes. These complexes are then allowed to contact a receptive material (e.g., antibodies) immobilized within the detection zone. Binding occurs between the analyte/probe conjugate complexes and the immobilized receptive material, thereby localizing “sandwich” complexes that are detectable to indicate the presence of the analyte or antigen on any one of the cells disclosed herein. This technique may be used to obtain quantitative or semi-quantitative results. Some examples of such sandwich-type assays are described by U.S. Pat. No. 4,168,146.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. Hybridization is carried out in conditions permitting specific hybridization. The length of the complementary sequences, the secondary structure, and GC content affect the thermal melting point Tₘ of the hybridization conditions necessary for obtaining specific hybridization of the target site to the target nucleic acid. Hybridization may be carried out under stringent conditions. The phrase “stringent hybridization conditions” refers to conditions under which a probe will hybridize to its target subsequence, typically in a complex mixture of nucleic acid, but to no other sequences at a detectable or significant level. Stringent conditions are sequence-dependent and will be different in different circumstances. Stringent conditions are those in which the salt concentration is less than about 1.0 M sodium ion, such as less than about 0.01 M, including from about 0.001 M to about 1.0 M sodium ion concentration (or other salts) at a pH between about 6 to about 8 and the temperature is in the range of about 20° C. to about 65° C. Stringent conditions may also be achieved with the addition of destabilizing agents, such as but not limited to formamide.

The oligonucleotide sequences, nucleic acid sequences, or other agents of the present disclosure can be administered, inter alia, as pharmaceutically acceptable salts, esters, or amides. The term “salts” refers to inorganic and organic salts of compounds of the present disclosure. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977). In some embodiments, the compositions disclosed herein comprise one or a plurality of salts of the oligonucleotide sequences disclosed herein.

The terms “thermal melting point”, “melting temperature” or “Tₘ” refer herein to the temperature (under defined ionic strength, pH, and nucleic acid concentration) at which 50% of probes complementary to a target hybridize to the target sequence at equilibrium (as the target sequences are present in excess, at Tₘ, 50% of the probes are occupied at equilibrium). In some cases, the term “T_{d}” is used to define the temperature at which at least half of a probe dissociates from a perfectly matched target nucleic acid.

The formation of a duplex molecule with all perfectly formed hydrogen-bonds between corresponding nucleotides is referred as “matched” or “perfectly matched”, and duplexes with single or several pairs of nucleotides that do not correspond are referred to as “mismatched.” Any combination of single-stranded RNA or DNA molecules can form duplex molecules (DNA:DNA, DNA:RNA, RNA:DNA, or RNA:RNA) under appropriate experimental conditions. Similarly, synthetic analogs can form duplex molecules with each other or RNA and DNA under the appropriate conditions.

The phrase “selectively (or specifically) hybridizing” refers to the binding, duplexing, or hybridizing of a molecule only to a particular nucleotide sequence under stringent hybridization conditions when that sequence is present in a complex mixture (e.g. total cellular or library DNA or RNA). Those of ordinary skill in the art will readily recognize that alternative hybridization and wash conditions can be utilized to provide conditions of similar stringency and will recognize that the combination of parameters is much more important than the measure of any single parameter.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

The term “substantially similar” as used in the context of nucleic acid or amino acid sequence identity refers to two or more sequences which have at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity.

As used herein “% sequence identity” is determined using the EMBOSS Pairwise Alignment Algorithms tool available from The European Bioinformatics Institute (EMBL-EBI), which is part of the European Molecular Biology Laboratory (EMBL). This tool is accessible at the website located by placing “www,” in front of “ebi.ac.uk/Tools/emboss/align/”. This tool utilizes the Needleman-Wunsch global alignment algorithm (Needleman, S. B. and Wunsch, C. D. (1970) J. Mol. Biol. 48, 443-453; Kruskal, J. B. (1983) An overview of sequence comparison In D. Sankoff and B. Kruskal, (ed.), Time warps, string edits and macromolecules: the theory and practice of sequence comparison, pp. 1-44 Addison Wesley. Default settings are utilized which include Gap Open: 10.0 and Gap Extend 0.5. The default matrix “Blosum62” is utilized for amino acid sequences and the default matrix “DNAfull” is utilized for nucleic acid sequences.

The terms “operably linked” refers to a juxtaposition of two or more components (such as sequence elements), in which the components are arranged such that both components function normally and allow the possibility that at least one of the components can mediate a function that is exerted upon at least one of the other components.

“Barcode” as used herein refers to a sequence, tag or combination of tags associated with a polynucleotide the identity of which (e.g., the tag DNA sequence) can be used to differentiate polynucleotides in a sample. In certain embodiments, the barcode on a polynucleotide is used to identify the source from which the polynucleotide is derived. For example, a nucleic acid sample may be a pool of polynucleotides derived from different sources, (e.g., polynucleotides derived from different individuals, different tissues or cells, or polynucleotides isolated at different times points), where the polynucleotides from each different source are tagged with a unique barcode. As such, a barcode provides a correlation between a polynucleotide and its source. In certain embodiments, barcodes are employed to uniquely tag each individual polynucleotide in a sample. Identification of the number of unique barcodes in a sample can provide a readout of how many individual polynucleotides are present in the sample (or from how many original polynucleotides a manipulated polynucleotide sample was derived; see, e.g., U.S. Pat. No. 7,537,897, incorporated herein by reference in its entirety). Barcodes can range in length from about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nucleotide bases or more and may include multiple subunits, where each different barcode has a distinct identity and/or order of subunits. Exemplary nucleic acid tags that find use as barcodes are described in U.S. Pat. No. 7,544,473, as well as U.S. Pat. No. 7,393,665, both of which are incorporated herein by reference in their entirety for their description of nucleic acid tags and their use in identifying oligonucleotides. In certain embodiments, a set of barcodes employed to tag a plurality of samples need not have any particular common property (e.g., Tₘ, length, base composition, etc.), as the methods described herein can accommodate a wide variety ofunique barcode sets. It is emphasized here that barcodes need only be unique within a given experiment. Thus, the same barcode may be used to tag a different sample being processed in a different experiment. In addition, in certain experiments, a user may use the same barcode to tag a subset of different samples within the same experiment. For example, all samples derived from individuals having a specific phenotype may be tagged with the same barcode, e.g., all samples derived from control (or wild type) subjects can be tagged with a first barcode while subjects having a disease condition can be tagged with a second barcode (different than the first barcode). As another example, it may be desirable to tag different samples derived from the same source with different barcodes (e.g., samples derived over time or derived from different sites within a tissue). Further, barcodes can be generated in a variety of different ways, e.g., by a combinatorial tagging approach in which one barcode is attached by ligation and a second barcode is attached by primer extension. In some embodiments, multiple unique barcodes can be attached to the same sample so as to increase its uniqueness with respect to other samples. Alternatively, one barcode could represent a class of samples (e.g., a well plate), and a second or third barcode could represent a specific well within that plate. Samples can be tagged, in some embodiments, with multiple barcodes by hybridizing more than one barcode oligonucleotide to a lipid-modified or hydrophobic-anchored oligonucleotide, or samples can be labeled with multiple barcoded lipid-modified or hydrophobic-anchored oligonucleotides. In some embodiments, individual cells could be barcoded via split-pool labeling to generate a unique barcode profile that is distinct from every other cell in the pool. Thus, barcodes can be designed and implemented in a variety of different ways to track polynucleotide fragments during processing and analysis, and thus no limitation in this regard is intended.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer sites, such reaction comprising one or more repetitions of the following steps: (i) denaturing the target nucleic acid, (ii) annealing primers to the primer sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“TAQMAN®”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al., Anal. Biochem. 273:221-28 (1999) (two-color real-time PCR). Usually, distinct sets of primers are employed for each sequence being amplified.

The term “hyperproliferative cell” means a cell that is cancerous, pre-cancerous, hyperplastic, or senescent and unable to proceed through mitosis normally. In some embodiments, the hyperproliferative cell is a tumor cell. In some embodiments, the hyperproliferative cell comprises a dysfunctional cell cycle rendering it deficient in apoptosis or metabolically unstable such that the cell proliferates faster than a cell of the same type and metabolically stable.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence or oligo portion of the nucleotide upon which the sequence is derived.

### Lipid-Modified Oligonucleotides

The disclosure relates to a composition and a method of using the composition for a cell barcoding method that uses recently developed specific sets of lipid-conjugated or hydrophobic-anchored oligonucleotides to efficiently label single cells derived from distinct patients or test conditions. Oligonucleotide barcodes (engineered with a PCR handle, unique identifier and capture sequence) can be subsequently introduced to the cells and subsets of the cells processed for droplet microfluidics-based RNA sequencing library preparation.

Stably embedding lipid-modified oligonucleotides within the cellular plasma membrane via a two-component system is disclosed in Selden et al., J. Am. Chem. Soc. 134:765-68 (2012); Weber et al., BioMacromolecules 15:4621-26 (2014); and Published U.S. Patent Application No. 2017/0305955, each of which incorporated herein by reference in their entireties.

One general and non-limiting example of a lipid-modified oligonucleotide as disclosed herein is presented in the claims. This lipid-modified oligonucleotide comprises three oligonucleotides: a first oligonucleotide comprising, from a 5′ to 3′ orientation, a first lipid moiety, a first hybridization region, and a first primer region; a second oligonucleotide comprising, from a 5′ to a 3′ orientation, a second hybridization region and a second lipid moiety, wherein the second hybridization region is the reverse complement of the first hybridization region; and a third oligonucleotide comprising, from a 5′ to a 3′ orientation, a second primer region, a barcode region, and a capture sequence, wherein the second primer region is the reverse complement of the first primer region.

The present disclosure also relates to microfluidics and labeled nucleic acids. For example, certain aspects are generally directed to systems and methods for labeling nucleic acids within microfluidic droplets or other compartments, for instance, arising from a cell. In one set of embodiments, particles may be prepared containing oligonucleotides that can be used to determine target nucleic acids, e.g., attached to the surface of the particles. The oligonucleotides may include “barcodes” or unique sequences that can be used to distinguish nucleic acids in a droplet from those in another droplet, for instance, even after the nucleic acids are pooled together or removed from the droplets. Certain embodiments of the invention are generally directed to systems and methods for attaching additional or arbitrary sequences to the nucleic acids within microfluidic droplets or other compartments, e.g., recognition sequences that can be used to selectively determine or amplify a desired sequence suspected of being present within a droplet. Such systems may be useful, for example, for selective amplification in various applications, such as high-throughput sequencing applications.

Some aspects of the present disclosure are generally directed to systems and methods for containing or encapsulating nucleic acids with lipid-modified or hydrophobic-anchored oligonucleotides within microfluidic droplets or other suitable compartments, for example, microwells of a microwell plate, individual spots on a slide or other surface, or the like. The nucleic acids and the oligonucleotides may be ligated or attached together in some cases. The nucleic acids may arise from lysed cells, organelles, or other material within the droplets. The oligonucleotides within a droplet may be distinguishable from oligonucleotides in other droplets, e.g., within a plurality or population of droplets. For instance, the oligonucleotides may contain one or more unique sequences or “barcodes” that are different between the various droplets. Thus, the nucleic acid within each droplet can be uniquely identified by determining the barcodes associated with the nucleic acid. This may be important, for example, if the droplets are “broken” or ruptured and the nucleic acids from different droplets are subsequently combined or pooled together, e.g., for sequencing or other analyses.

The disclosure relates to a cell comprising one or a plurality of lipid-modified oligonucleotides, wherein the lipid-modified oligonucleotide comprises a lipid moiety region and, optionally, a capture regions. In some embodiments, the cell is a hyperproliferative cell, a transformed cell from a cell line, or a primary cell isolated from a subject or patient.

The disclosure also relates to a system comprising a plate, dish or other solid support comprising one or a plurality of vessels into which samples, biopsies, cells or tissues are immobilized or absorbed to the vessel surface and exposed one or a plurality of barcodes, each vessel comprising a unique barcode or plurality of barcodes corresponding to the cells and/or location of expression of nucleic acids in the samples, biopsies, cells or tissues.

### Lipid Moiety Regions

In some embodiments, the lipid moiety region comprises an alkyl chain and an alkenyl, alkyl, aryl, or aralkyl chain. This alkenyl, alkyl, aryl, or aralkyl chain may comprise about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 carbon atoms or more. In some embodiments, the alkyl chain comprises about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 carbon atoms or more, and the alkenyl, alkyl, aryl, or aralkyl chain comprises about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 carbon atoms or more. In some embodiments, the chains share the same number of carbon atoms. In other embodiments, one chain has between about 1, 2, 3, 4, 5, 6, 7, 8, 9, or 10 fewer carbon atoms than the other chain. The lipid moiety region may comprise more than one alkenyl, aryl, or aralkyl chain, with each chain comprising 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 carbon atoms or more.

In some embodiments, the lipid moiety region may contain one or more unsaturated carbon bonds. In some embodiments, the unsaturated bonds are all contained within the same chain. In still other embodiments, the unsaturated bonds may be contained in more than one chain.

In certain embodiments, the lipid moiety region comprises a dialkylphosphoglycieride, and the polynucleotide is conjugated to the dialkylphosphoglycieride. In some embodiments, each chain of the dialkylphosphoglycieride has the same number of carbon atoms with the other chain. In other embodiments, the number of carbon atoms is different between the two alkyl chains of the dialkylphosphoglycieride. In some embodiments, each chain has 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 carbon atoms or more. In some embodiments, each chain has about 12 carbon atoms, or about 14 carbon atoms, about 16 carbon atoms, about 18 carbon atoms, about 20 carbon atoms, or about 22 carbon atoms. In some embodiments, at least one chain has about 12 carbon atoms, about 14 carbon atoms, about 16 carbon atoms, about 18 carbon atoms, about 20 carbon atoms, or about 22 carbon atoms.

The lipid moiety region may comprise a monoalkylamide, and the polynucleotide may be conjugated to the monoalkylamide. In some embodiments, the monoalkylamide chain has about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 carbon atoms or more. In some embodiments, the monoalkylamide chain has about 12 carbon atoms, or about 14 carbon atoms, about 16 carbon atoms, about 18 carbon atoms, about 20 carbon atoms, or about 22 carbon atoms. In certain embodiments, the monoalkylamide comprises about 16 or 18 carbon atoms.

In other embodiments, the lipid moiety region and the polynucleotide are joined by a compound comprising, a phosphate group. In other embodiments, the lipid moiety region and the polynucleotide are joined by a compound comprising a urea group. In still other embodiments, the lipid moiety region and the polynucleotide are joined by a compound comprising a sulfonyl group. In another embodiment, the lipid moiety region and the polynucleotide are joined by a compound comprising a sulfonamide, ether, thioether, carbamate, or carbonate group.

In still other embodiments, the lipid moiety region may comprise a sterol group. In some embodiments, the sterol group may be natural or synthetic or derived from a sterol compound bearing (or modified to bear) a functional group used for attachment to the polynucleotide. For instance, sterols from biological sources are usually found either as free sterol alcohols, acylated (sterol esters), alkylated (steryl alkyl ethers), sulfated (cholesterol sulfate), or linked to a glycoside moiety (steryl glycosides) which can be itself acylated (acylated sterol glycosides) (See, e.g., Fahy et al., J. Lipid Res. 46:839-61 (2005), which reference is incorporated in its entirety). Examples include (1) sterols obtainable from animal sources, referred to herein “zoosterols” such as the zoosterols cholesterol and certain steroid hormones; and (2) sterols obtainable from plants, fungi and marine sources, referred to herein as “phytosterols,” such as the phytosterols campesterol, sitosterol, stigmasterol, and ergosterol. These sterols generally bear at least one free hydroxyl group, usually at the 3 position of ring A, at another position, or combinations thereof, or can be modified to incorporate a suitable hydroxyl or other functional group as needed.

Sterols of particular interest are the simple sterols, which bear a unique functional group for attachment to the polynucleotide. Of specific interest are simple sterols in which the unique functional group is a hydroxyl, and in particular, the simple sterol alcohols having a hydroxyl group located at position 3 of ring A (e.g., cholesterol, .beta.-sitosterol, stigmasterol, campesterol, and brassicasterol, ergosterol and the like, and derivatives thereof).

Cholesterol is of particular interest in certain embodiments for inclusion in the lipid moiety region. Representative sterols of the cholesterol class (including substituted cholesterols) of interest include, for example, the following: (1) natural and synthetic sterols such as cholesterol (ovine wool), cholesterol (plant derived), desmosterol, stigmasterol, β-sitosterol, thiocholesterol, 3-cholesteryl acrylate; (2) A-ring substituted oxysterols such as cholestanol, and cholestenone; (3) B-ring substituted oxysterols such as 7-ketocholesterol, 5α,6α-epoxycholestanol, 50,60-epoxycholestanol, and 7-dehydrocholesterol; (4) D-ring substituted oxysterols such as 25-ketocholestene, and 15-ketocholestane; (5) side-chain substituted oxysterols such as 25-hydroxycholesterol, 27-hydroxycholesterol, 24(R/S)-hydroxycholesterol, 24(R/S),25-epoxycholesterol, and 24(S),25-epoxycholesterol; (6) lanosterols such as 24-dihydrolanosterol and lanosterol; (7) fluorinated sterols such as F7-cholesterol, F7-5α,6α-epoxycholestanol, F7-5β,6β-epoxycholostanol, and F7-7-ketocholesterol; (8) fluorescent cholesterol such as 25-NBD cholesterol, dehydroergosterol, and cholesterol triene. These compounds may also include deuterated and non-deuterated versions, and are available commercially, such as from Avanti Polar Lipids, Inc.

In certain embodiments, the lipid moiety region may comprise a saturated or unsaturated, linear or branched, substituted or unsubstituted aliphatic chain. Of particular interest are saturated or unsaturated, linear or branched, substituted or unsubstituted hydrocarbon chains having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 carbons.

Further embodiments may comprise elements based on or derivable from various lipids, such the aliphatic acids, gycerolipids, glycerophospholipids, sphingolipids, prenol lipids, polyprenol lipids, and saccharolipids, such as the from lipids described in Fahy et al., J. Lipid Res. 46:839-61 (2005).

An “anchor” lipid-modified or hydrophobic-anchored oligonucleotide (e.g., a lipid-modified oligonucleotide comprising, from a 5′ to 3′ orientation or from a 3′ to 5′ orientation, a first lipid moiety, a first hybridization region, and a first primer region) and a “co-anchor” lipid-modified or hydrophobic-anchored oligonucleotide (e.g., a lipid-modified oligonucleotide comprising, from a 5′ to a 3′ orientation or from a 3′ to 5′ orientation, a second hybridization region and a second lipid moiety, wherein the second hybridization region is the reverse complement of the first hybridization region) can comprise the same lipid moiety or a different lipid moiety (e.g., different carbon chain lengths, different compositions, or different modifications). In some embodiments, the “anchor” lipid-modified or hydrophobic-anchored oligonucleotide comprises a lipid moiety that containing the same number of carbons as the lipid moiety of the “co-anchor” lipid-modified or hydrophobic-anchored oligonucleotide. In some embodiments, the “anchor” lipid-modified or hydrophobic-anchored oligonucleotide comprises a lipid moiety that contains about 1, 2, 3, 4, 5, 6, 7, 8, 9, 10, 11, or 12 or more carbons as compared to the lipid moiety of the “co-anchor” lipid-modified or hydrophobic-anchored oligonucleotide. In some embodiments, the “co-anchor” lipid-modified or hydrophobic-anchored oligonucleotide comprises a lipid moiety that contains about 1, 2, 3, 4, 5, 6, 7, 8, 9, 10, 11, or 12 or more carbons as compared to the lipid moiety of the “anchor” lipid-modified or hydrophobic-anchored oligonucleotide. In some embodiments, the “anchor” lipid-modified or hydrophobic-anchored oligonucleotide comprises a lipid moiety that contains about 1, 2, 3, 4, 5, 6, 7, 8, 9, 10, 11, or 12 or more carbons as compared to the lipid moiety of the “co-anchor” lipid-modified or hydrophobic-anchored oligonucleotide. In some embodiments, only an anchor lipid-modified or hydrophobic-anchored oligonucleotide is used without a corresponding co-anchor lipid-modified or hydrophobic-anchored oligonucleotide.

In some embodiments, the lipid moiety (i.e., the lipid moiety in embodiments with only an anchor lipid-modified or hydrophobic-anchored oligonucleotide or either the first or second lipid moiety in embodiments with both an anchor lipid-modified or hydrophobic-anchored oligonucleotide and a co-anchor lipid-modified or hydrophobic-anchored oligonucleotide) comprises a compound of Formula I:

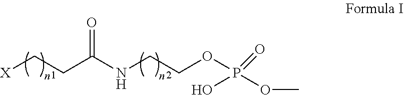

or a physiologically acceptable salt thereof,
wherein n¹ is from 5 to 25, n² is from 1 to 25, and X is selected from the group consisting of NH, CH₂, O, and CH—R, wherein R is a C12 to C28 monoglyceride, alkenyl, alkyl, aryl, or aralkyl.

In some embodiments, the lipid moiety (i.e., the lipid moiety in embodiments with only an anchor lipid-modified or hydrophobic-anchored oligonucleotide or either the first or second lipid moiety in embodiments with both an anchor lipid-modified or hydrophobic-anchored oligonucleotide and a co-anchor lipid-modified or hydrophobic-anchored oligonucleotide) comprises a compound of Formula II:

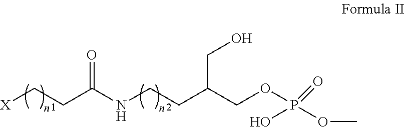

or a physiologically acceptable salt thereof,
wherein n¹ is from 5 to 25, n² is from 0 to 24, and X is selected from the group consisting of NH, CH₂, O, and CH—R, wherein R is a C12 to C28 monoglyceride, alkenyl, alkyl, aryl, or aralkyl.

In some embodiments, the lipid moiety, first lipid moiety, the second lipid moiety, or both lipid moieties comprises a compound of Formula III:

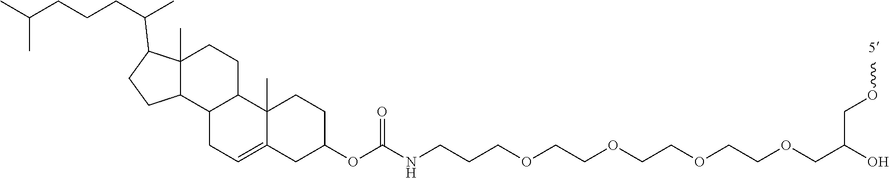

In some embodiments, the “anchor” lipid-modified or hydrophobic-anchored oligonucleotide comprises a sterol moiety and the “co-anchor” lipid-modified or hydrophobic-anchored oligonucleotide comprises a lipid moiety. In some embodiments, the “co-anchor” lipid-modified or hydrophobic-anchored oligonucleotide comprises a sterol moiety and the “anchor” lipid-modified or hydrophobic-anchored oligonucleotide comprises a lipid moiety. In some embodiments, both the “anchor” lipid-modified or hydrophobic-anchored oligonucleotide and the “co-anchor” lipid-modified or hydrophobic-anchored oligonucleotide comprises a sterol moiety.

### Hybridization Regions

Anchor lipid-modified or hydrophobic-anchored oligonucleotides and co-anchor lipid-modified or hydrophobic-anchored oligonucleotides comprise hybridization regions that are complementary to each other. An anchor lipid-modified or hydrophobic-anchored oligonucleotide comprises the lipid moiety operably linked (e.g., covalently linked) to a first hybridization region comprising an oligonucleotide of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more nucleotide bases. The oligonucleotide can be DNA, RNA, or modified or synthetic DNA or RNA.

The co-anchor lipid-modified or hydrophobic-anchored oligonucleotides comprise the lipid moiety operably linked (e.g., covalently linked) to a second hybridization region comprising an oligonucleotide of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more nucleotide bases. The oligonucleotide can be DNA, RNA, or modified or synthetic DNA or RNA. In some embodiments, the second hybridization region is the same type of nucleic acid as the first hybridization region (e.g., if the first hybridization region is DNA, then the second hybridization region is DNA), or the second hybridization region can be a different type of nucleic acid compared to the first hybridization region (e.g., if the first hybridization region is DNA, then the second hybridization region could be RNA, or modified or synthetic DNA or RNA).

The second hybridization region is a reverse complement of the first hybridization region. In some embodiments, the complementarity can be perfect complementarity (i.e., the second hybridization region is the same length as the first hybridization region and each base of the second hybridization region is a perfect complement to the its base pair on the first hybridization region). In some embodiments, the first hybridization region contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 or more additional bases than the second hybridization region. In some embodiments, the second hybridization region contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 or more additional bases than the first hybridization region. In some embodiments, the first hybridization region has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the second hybridization region.

In some embodiments, the first hybridization region has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 4 (GTAACGATCCAGCTGTCACT).

In some embodiments, the second hybridization region has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 2 (AGTGACAGCTGGATCGTTAC).

### Primer Regions

Anchor lipid-modified or hydrophobic-anchored oligonucleotides and barcode oligonucleotides comprise primer regions that are complementary to each other. An anchor lipid-modified or hydrophobic-anchored oligonucleotide comprises the lipid moiety operably linked (e.g., covalently linked) to the first hybridization region which is operably linked (e.g., covalently linked) to a first primer region comprising an oligonucleotide of 10, 11, 12, 13, 14, 15, 16, 17, 18, 19, 20, 21, 22, 23, 24, 25, 26, 27, 28, 29, or 30 or more nucleotide bases. The oligonucleotide can be DNA, RNA, or modified or synthetic DNA or RNA.

The barcode oligonucleotides comprise a second primer region operably linked (e.g., covalently linked) to a barcode region (described below) which in turn is operably linked (e.g., covalently linked) to a capture sequence (described below), the second primer region comprising an oligonucleotide of 10, 11, 12, 13, 14, 15, 16, 17, 18, 19, 20, 21, 22, 23, 24, 25, 26, 27, 28, 29, or 30 or more nucleotide bases. The oligonucleotide can be DNA, RNA, or modified or synthetic DNA or RNA. In some embodiments, the second primer region is the same type of nucleic acid as the first primer region (e.g., if the first primer region is DNA, then the second primer region is DNA), or the second primer region can be a different type of nucleic acid compared to the first primer region (e.g., if the first primer region is DNA, then the second primer region could be RNA, or modified or synthetic DNA or RNA).

The second primer region is a reverse complement of the first primer region. In some embodiments, the complementarity can be perfect complementarity (i.e., the second primer region is the same length as the first primer region and each base of the second primer region is a perfect complement to the its base pair on the first primer region). In some embodiments, the first primer region contains 1, 2, 3, 4, 5, 6, 7, 8, 9, 10, 11, 12, 13, 14, 15, 16, 17, 18, 19, or 20 or more additional bases than the second primer region. In some embodiments, the second primer region contains 1, 2, 3, 4, 5, 6, 7, 8, 9, 10, 11, 12, 13, 14, 15, 16, 17, 18, 19, or 20 or more additional bases than the first primer region. In some embodiments, the first primer region has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the second primer region.

In some embodiments, the first primer region has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5 (TGGAATTCTCGGGTGCCAAGG).

In some embodiments, the second primer region has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 6 (CCTTGGCACCCGAGAATTCCA).

### Barcode Regions

The barcode oligonucleotides comprise the second primer region operably linked (e.g., covalently linked) to a barcode region which in turn is operably linked (e.g., covalently linked) to a capture sequence (described below), the barcode region comprising an oligonucleotide of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more nucleotide bases. The oligonucleotide can be DNA, RNA, or modified or synthetic DNA or RNA. Methods of designing sets of barcode sequences are shown for example in U.S. Pat. No. 6,235,475, the contents of which are incorporated by reference herein in their entirety. Attaching barcode sequences to nucleic acid templates is shown in U.S. Pub. 2008/0081330 and U.S. Pub. 2011/0301042, the content of each of which is incorporated by reference herein in its entirety. Methods for designing sets of barcode sequences and other methods for attaching barcode sequences are shown in U.S. Pat. Nos. 6,138,077; 6,352,828; 5,636,400; 6,172,214; 6,235,475; 7,393,665; 7,544,473; 5,846,719; 5,695,934; 5,604,097; 6,150,516; RE39,793; 7,537,897; 6,172,218; and 5,863,722, the content of each of which is incorporated by reference herein in its entirety. Barcodes for sequencing and copy number estimation are described in U.S. Pub. 2016/0046986, incorporated herein by reference in its entirety.

Barcodes can be completely random or they can be engineered with certain predetermined sequences. They may have regions of randomness or semi-randomness and other fixed regions. The barcodes may include other regions, such as priming sites, adapters, or other complimentary regions that would facilitate further processing and analysis. The identity of a specific barcode itself in relation to its second primer region can be created prior to any binding or capture of an anchor lipid-modified or hydrophobic-anchored oligonucleotide through hybridization of the first and second primer regions. Thus, in some embodiments, a database of all barcodes is created and stored, in some embodiments, on a computer storage medium.

In some embodiments, the last nucleotide of the barcode region will not be identical to the first nucleotide of the capture sequence. For example, if the capture sequence is a polyadenylation tail, the last nucleotide of the barcode region, in some embodiments, will not be adenine.

The barcode enables tagging or tracking of cells or membranes comprising the lipid-modified or hydrophobic-anchored oligonucleotides in order to permit subsequent identification and origin of the particular cell or membrane. The assignment of a barcode to individual or subgroups of oligonucleotides may allow for a unique identity to be assigned to individual sequences, fragments of sequences, or cells. This may allow acquisition of data from individual samples and is not limited to averages of samples.

In some embodiments, oligonucleotides may share a common barcode and therefore may be later identified as being derived from the same target cell. Multiple cells (of the same or different types) can be identified through use of multiple barcodes, with each barcode identifying a specific cell type or multiple cells within a specific cell type.

A single cell or membrane could comprise more than one lipid-modified or hydrophobic-anchored oligonucleotide, with each lipid-modified or hydrophobic-anchored oligonucleotide having a different first primer region. Such cells or membranes could thus be isolated and/or identified through different barcode oligonucleotides, each comprising a second primer region having complementarity to a different first primer region, and a single barcode for each barcode oligonucleotide or different barcodes for some or all of the barcode oligonucleotides.

### Capture Sequence

In some embodiments, the barcode oligonucleotides comprise the second primer region operably linked (e.g., covalently linked) to the barcode region which in turn is operably linked (e.g., covalently linked) to a capture sequence (described below), the capture sequence comprising an oligonucleotide of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more nucleotide bases. The oligonucleotide can be DNA, RNA, or modified or synthetic DNA or RNA.

In some embodiments, the capture sequence is a polyadenylated tail (a “poly(A) tail”), that is, the entire capture sequence consists of adenine bases. In some embodiments, the capture sequence has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a poly(A) tail. In some embodiments, the capture sequence has the sequence of SEQ ID NO: 7 (AAAAAAAAAAAAAAAAAAAAAAAAAAAAAAAA).

In some embodiments, the capture sequence is a polythymine tail (a “poly(T) tail”), that is, the entire capture sequence consists of thymine bases. In some embodiments, the capture sequence has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a poly(T) tail.

In some embodiments, the capture sequence is a polyuracil tail (a “poly(U) tail”), that is, the entire capture sequence consists of uracil bases. In some embodiments, the capture sequence has at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to a poly(U) tail.

In some embodiments, the capture sequence is a variant of a poly(A), poly(T), or poly(U) tail. Such variants include bases besides a pure poly(A), poly(T), or poly(U) tail. For example, variants can include capture sequences such as poly(A) variants like AAUAAA, AUUAAA AACAAG, AACAAA, AAUAAU, AAUAAG, UAUAAA, AGUAAA, AAUACA, CAUAAA, AAUAUA, GAUAAA, AAUGAA, AAGAAA, ACUAAA, AAUAGA, AAUAAU, AACAAA, AUUACA, AUUAUA, AACAAG, or AAUAAG, with each variant containing additional nucleotide bases if needed, e.g. 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 additional nucleotide bases, with the majority, and in some embodiments all, of the additional nucleotide bases being adenine. Variant poly(T) and poly(U) capture sequences can be similarly constructed.

In some embodiments, instead of a capture sequence, a member of a coupling pair (such as, e.g., antibody/antigen, receptor/ligand, or the avidin-biotin pair as described in, e.g., U.S. Patent Application No. 2006/0252077, incorporated herein by reference) may be linked to each fragment to be captured on a surface coated with a respective second member of that coupling pair. Subsequent to the capture, the sequence may be analyzed, for example, by single molecule detection/sequencing, e.g., as described in U.S. Pat. No. 7,283,337, incorporated herein by reference.

### Methods of Synthesizing Lipid-Modified or Hydrophobic-Anchored Oligonucleotides

Oligonucleotides can be synthesized using protocols known in the art, for example as described in Caruthers et al., Meth. Enzymol. 211:3 (1992); WO 99/54459; Wincott et al., Nucleic Acids Res. 23:2677 (1995); Wincott et al., Meth. Mol. Bio. 74:59 (1997); Brennan et al., Biotechnol. Bioeng. 61:33 (1998); and U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end.

Oligonucleotides disclosed herein encompass native and synthetic or modified oligonucleotides. A modified nucleic acid has one or more modifications, e.g., a base modification, a backbone modification, etc., to provide the nucleic acid with a new or enhanced feature (e.g., improved stability). A nucleoside can be a base-sugar combination, the base portion of which is a heterocyclic base. Heterocyclic bases include the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In some cases, the respective ends of this linear polymeric compound can be further joined to form a circular compound. In addition, linear compounds may have internal nucleotide base complementarity and may therefore fold in a manner as to produce a fully or partially double-stranded compound. Within oligonucleotides, the phosphate groups can be referred to as forming the internucleoside backbone of the oligonucleotide. The linkage or backbone of RNA and DNA can be a 3′ to 5′ phosphodiester linkage.

Examples of suitable nucleic acids containing modifications include nucleic acids with modified backbones or non-natural internucleoside linkages. Nucleic acids having modified backbones include those that retain a phosphorus atom in the backbone and those that do not have a phosphorus atom in the backbone. Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Suitable oligonucleotides having inverted polarity include a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid has one or more phosphorothioate and/or heteroatom internucleoside linkages, in particular —CH₂—NH—O—CH₂—, —CH₂—N(CH₃)—O—CH₂-(known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in U.S. Pat. No. 5,602,240.

Also suitable are nucleic acids having morpholino backbone structures as described in, e.g., U.S. Pat. No. 5,034,506. For example, in some embodiments, a subject nucleic acid includes a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Also included are nucleic acid mimetics. The term “mimetic” as it is applied to polynucleotides encompasses polynucleotides where only the furanose ring or both the furanose ring and the internucleotide linkage are replaced with non-furanose groups, replacement of only the furanose ring is also referred to as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of suitable polynucleotide mimetic is based on linked morpholino units (morpholino nucleic acid) having heterocyclic bases attached to the morpholino ring. A number of linking groups have been reported that can link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Braasch et al., Biochemistry, 41(14):4503-10 (2002)). Morpholino-based polynucleotides are disclosed in U.S. Pat. No. 5,034,506. A variety of compounds within the morpholino class of polynucleotides have been prepared, having a variety of different linking groups joining the monomeric subunits.

Another suitable class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 122:8595-8602 (2000)). The incorporation of CeNA monomers into a DNA chain increases the stability of a DNA/RNA hybrid. CeNA oligoadenylates formed complexes with RNA and DNA complements with similar stability to the native complexes. The incorporation CeNA structures into natural nucleic acid structures was shown by NMR and circular dichroism to proceed with conformational adaptation.

Also suitable as modified nucleic acids are Locked Nucleic Acids (LNAs) and/or LNA analogs. In an LNA, the 2′-hydroxyl group is linked to the 4′ carbon atom of the sugar ring thereby forming a 2′-C,4′-C-oxymethylene linkage, and thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 4:455-456 (1998)). LNA and LNA analogs display very high duplex thermal stabilities with complementary DNA and RNA (Tm=+3 to +10° C.), stability towards 3′-exonucleolytic degradation and good solubility properties. Potent and nontoxic oligonucleotides containing LNAs have been described (Wahlestedt et al., Proc. Natl. Acad. Sci. U.S.A., 97:5633-38 (2000)).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 54:3607-30 (1998)). LNAs and preparation thereof are also described in WO98/39352 and WO99/14226, both of which are hereby incorporated by reference in their entirety. Exemplary LNA analogs are described in U.S. Pat. Nos. 7,399,845 and 7,569,686, both of which are hereby incorporated by reference in their entirety.

A nucleic acid can also include one or more substituted sugar moieties. Suitable polynucleotides include a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Also suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides include a sugar substituent group selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, and other substituents having similar properties. A suitable modification can include 2′-methoxyethoxy (2′-O—CH₂CH₂OCH₃, also known as 2′-O-(2-methoxyethyl) or 2′-MOE) (Martin et al., Helv. Chim. Acta, 78:486-504 (1995)) i.e., an alkoxyalkoxy group. A suitable modification can include 2′-dimethylaminooxyethoxy, i.e., a O(CH₂)₂ON(CH₃)₂ group, also known as 2′-DMAOE, and 2′-dimethylaminoethoxyethoxy (also referred to as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

A nucleic acid may also include a nucleobase (also referred to as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Modified nucleobases also include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), and pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

Lipids (e.g., lipid, lipids, lipid precursors, lipid precursor, oleochemicals, or oleochemical) can be produced by any chemical or biochemical (e.g., as found in U.S. Pat. Nos. 9,896,691, 9,598,710, 9,499,829, 9,428,779, 9,127,288, incorporated herein by reference in their entireties; and Kinney, 1997, Genetic Engeneering, Ed.: J K Setlow, 19:149-166; Ohlrogge and Browse, 1995, Plant Cell 7:957-970; Shanklin and Cahoon, 1998, Annu. Rev. Plant Physiol. Plant Mol. Biol. 49:611-641; Voelker, 1996, Genetic Engineering, Ed.: J K Setlow, 18:111-13; Gerhardt, 1992, Prog. Lipid R. 31:397-417; Guhnemann-Schafer & Kindl, 1995, Biochim. Biophys Acta 1256:181-186; Kunau et al., 1995, Prog. Lipid Res. 34:267-342; Stymne et al., 1993, in: Biochemistry and Molecular Biology of Membrane and Storage Lipids of Plants, Ed.: Murata and Somerville, Rockville, American Society of Plant Physiologists, 150-158, Murphy & Ross 1998, Plant Journal. 13(1):1-16) and collected by any convenient method (e.g. centrifugation of extracellular secreted lipids, exposure to solvent, whole cell extraction (e.g. cell disruption and collection), hydrophobic solvent extraction (e.g. hexane), liquefaction, supercritical carbon dioxide extraction, freeze drying, mechanical pulverization, secretion (e.g. by addition of effective exporter proteins), or combinations thereof). In some embodiments, the lipids can be extracted and purified from, e.g., a plant, bacteria, or oleaginous yeast or fungi.

In some embodiments, lipids are covalently linked to the oligonucleotides disclosed herein. In some embodiments, lipids are cross-linked to the oligonucleotides disclosed herein. The manner of binding the lipids to the oligonucleotides is not particularly limited. The lipids and the oligonucleotides may be bound directly or via a linker (a linkage region). In some embodiments, the linker used to bind the lipids to the oligonucleotides comprises a nucleic acid. In some embodiments, the linker used to bind the lipids to the oligonucleotides does not comprise a nucleic acid.

The linker that can be used is not particularly limited insofar as the lipids and the oligonucleotide are covalently linked to each other. Examples of usable linkers include those of the following structures: —O—P(═O)(OH)—O—, —O—CO—O—, —NH—CO—O—, —NH—CO—NH—, —NH—(CH₂)ₙ₁—, —S—(CH₂)ₙ₁—, —CO—(CH₂)ₙ₁—CO—, —CO—(CH₂)ₙ₁—NH—, —NH—(CH₂)ₙ₁—NH—, —CO—NH—(CH₂)ₙ₁—NH—CO—, —C(═S)—NH—(CH₂)ₙ₁—NH—CO—, —C(═S)—NH—(CH₂)ₙ₁—NH—C—(═S)—, —CO—O—(CH₂)ₙ₁—O—CO—, —C(═S)—O—(CH₂)ₙ₁—O—CO—, —C(═S)—O—(CH₂)ₙ₁—O—C—(═S)—, —CO—NH—(CH₂)ₙ₁—O—CO—, —C(═S)—NH—(CH₂)ₙ₁—O—CO—, —C(═S)—NH—(CH₂)ₙ₁—O—C—(═S)—, —CO—NH—(CH₂)ₙ₁—O—CO—, —C(═S)—NH—(CH₂)ₙ₁—CO—, —C(═S)—O—(CH₂)ₙ₁—NH—CO—, —C(═S)—NH—(CH₂)ₙ₁—O—C—(═S)—, —NH—(CH₂CH₂O)ₙ₂—CH(CH₂OH)—, —NH—(CH₂CH₂O)ₙ₂—CH₂—, —NH—(CH₂CH₂O)ₙ₂—CH₂—CO—, —O—(CH₂)ₙ₃—S—S—(CH₂)ₙ₄—O—P(═O)₂—, —CO—(CH₂)ₙ₃—O—CO—NH—(CH₂)ₙ₄—, —CO—(CH₂)ₙ₃—CO—NH—(CH₂)ₙ₄—, wherein n1 is an integer from about 1 to about 40, n2 is an integer from about 1 to about 20, and, n3 and n4 may be the same or different, and are an integer from about 1 to about 20. In some embodiments, the linker is a phosphate group (—O—P(═O)(OH)—O—).

### Methods of Use

Lipid-modified or hydrophobic-anchored oligonucleotide compounds and lipid-modified or hydrophobic-anchored oligonucleotide-containing compositions can be used in a variety of different pharmaceutical, cosmeceutical, diagnostic and biomedical applications. For example, lipid-modified or hydrophobic-anchored oligonucleotide compounds and compositions comprising lipid-modified or hydrophobic-anchored oligonucleotide compounds find use in research and therapeutic applications, including the study of cell-cell interactions, membrane mechanics, the bottom-up assembly of tissues, the quantitative imaging of non-adherent cells, or the study of biological processes occurring near a cell surface. Lipid-modified or hydrophobic-anchored oligonucleotide compounds and lipid-modified or hydrophobic-anchored oligonucleotide-containing compositions can also be used to study the spatial expression profile of a gene in a sample or tissue from a subject.

In practicing such methods, a composition comprising a lipid-modified or hydrophobic-anchored oligonucleotide may first be contacted with a membrane, such as a cell membrane, a plasma membrane dividing the cytoplasm from a point outside of the cell, or a nuclear membrane, under conditions allowing insertion of said composition into said membrane. In some embodiments, the method comprises contacting a membrane with a composition comprising a lipid-modified or hydrophobic-anchored oligonucleotide and incubating said composition with said membrane under conditions for allowing insertion of said composition into said membrane. In some embodiments, the method comprises contacting a membrane with a composition comprising a lipid-modified or hydrophobic-anchored oligonucleotide and incubating said composition with said membrane for a time period sufficient for anchor-lipid modified oligonucleotides to embed themselves within the membrane.

In some embodiments, anchor lipid-modified or hydrophobic-anchored oligonucleotides and co-anchor lipid-modified or hydrophobic-anchored oligonucleotides are added to cells or membranes simultaneously. In some embodiments, anchor lipid-modified or hydrophobic-anchored oligonucleotides and co-anchor lipid-modified or hydrophobic-anchored oligonucleotides are added to cells or membranes sequentially. In some embodiments, anchor lipid-modified or hydrophobic-anchored oligonucleotides are added to cells or membranes first followed by addition of co-anchor lipid-modified or hydrophobic-anchored oligonucleotides to the cells or membranes. In some embodiments, co-anchor lipid-modified or hydrophobic-anchored oligonucleotides are added to cells or membranes first followed by addition of anchor lipid-modified or hydrophobic-anchored oligonucleotides to the cells or membranes. In some embodiments, anchor lipid-modified or hydrophobic-anchored oligonucleotides are pre-hybridized to barcode oligonucleotides prior to being added to cells or membranes. In such embodiments, anchor lipid-modified or hydrophobic-anchored oligonucleotides are hybridized to barcode oligonucleotides through their respective primer regions.

In some embodiments, the disclosure relates to a method of labeling a cell or a cell sample, a method of isolating endogenous DNA from a cell sample, or a method of sequencing nucleic acid sequences from a cell sample comprising exposing the cell sample to one or a plurality of anchor-lipid modified oligonucleotides disclosed herein, then adding at least a first labeling oligonucleotide sequence complementary to the anchor-lipid modified oligonucleotide, the first labeling oligonucleotide sequence comprising a known nucleic acid sequence portion on its 3′ region. In some embodiments, the sample is from a human.

In some embodiments, the first labeling oligonucleotide is complementary to the 3′ region of the anchor-lipid modified oligonucleotide such that at least a portion of the known nucleic acid sequence of the 3′ end is single-stranded. In some embodiments, the method further comprises exposing the single-stranded 3′ end of the first labeling oligonucleotide to a ligase buffer and a ligase to covalently link the first labeling oligonucleotide to the anchor lipid-modified oligonucleotide. The anchor-lipid modified oligonucleotide may be exposed sequentially to at least a second, third or fourth or more labeling oligonucleotide, each of the first, second, third, fourth or more labeling oligonucleotides comprising a unique identifying nucleic acid sequence in their respective 3′ region of the molecule. In some embodiments, the method further comprises exposing the anchor lipid-modified oligonucleotides and the first or plurality of labeling oligonucleotides to a first linker, such oligonucleotide sequence complementary to a portion of the first labeling oligonucleotide and the second labeling oligonucleotide, such that, when exposed to ligase and free dNTPs, the first linker serves as a template nucleic acid strand for ligating and forming complementary nucleic acid sequence along a strand of nucleic acid of that is single-stranded region of the 3′ end of each labeling oligonucleotide. In some embodiments, the disclosure relates to a method of labeling a cell sample, a method of isolating endogenous DNA from a cell sample, or a method of sequencing nucleic acid sequences from a cell sample, the method comprises:

(a) exposing the cell sample to one or a plurality of anchor-lipid modified oligonucleotides disclosed herein or a composition comprising the same for a time period sufficient for the anchor-lipid modified oligonucleotide to embed itself within a cell membrane of the cell;

(b) exposing the cell sample to one or a plurality of labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides;

(c) ligating the one or plurality of labeling oligonucleotides to the one or plurality of anchor-lipid modified oligonucleotides; and, optionally

(d) detecting the presence of the one or plurality of labeling oligonucleotides by detection of one of more unique nucleotide sequences corresponding to the one or plurality of labeling oligonucleotides; and/or

(e) isolating the cell based upon the presence of one or plurality of labeling oligonucleotides, wherein the presence of the one or plurality of labeling oligonucleotides is determined by detection of one of more unique nucleotide sequences corresponding to the one or plurality of labeling oligonucleotides.

In some embodiments, the method of labeling a cell sample, the method of isolating endogenous DNA from a cell sample, or the method of sequencing nucleic acid sequences from a cell sample comprises:

(a) exposing a cell to one or a plurality of anchor-lipid modified oligonucleotides for a time period sufficient for the anchor-lipid modified oligonucleotide to embed itself within a cell membrane of the cell;

(b) exposing the cell to a first labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides;

(c) ligating the first labeling oligonucleotides to the one or plurality of anchor-lipid modified oligonucleotides; and, optionally

(d) detecting the presence of the first labeling oligonucleotides by detection of one of more unique nucleotide sequences corresponding to the first labeling oligonucleotides; and/or

(e) isolating the cell based upon the presence of the first labeling oligonucleotides, wherein the presence of the first labeling oligonucleotides is determined by detection of one of more unique nucleotide sequences corresponding to the first labeling oligonucleotides.

In some embodiments, the method of labeling a cell sample, the method of isolating endogenous DNA from a cell sample, or the method of sequencing nucleic acid sequences from a cell sample comprises:

(a) exposing a cell to one or a plurality of anchor-lipid modified oligonucleotides for a time period sufficient for the anchor-lipid modified oligonucleotide to embed itself within a cell membrane of the cell;

(b) exposing the cell to a first, second, third fourth or more labeling oligonucleotides, wherein the first labeling oligonucleotide is complementary to the one or a plurality of anchor-lipid modified oligonucleotides and exposed to the one or a plurality of anchor-lipid modified oligonucleotides for a time period sufficient for the anchor-lipid modified oligonucleotide to form a complementary strand of nucleic acid with the first labeling oligonucleotide, and wherein the second, third and/or fourth or more labeling oligonucleotides is exposed sequentially to a 3′ portion of the labeling oligonucleotide exposed to the cell immediately prior to the second, third and/or fourth or more labeling oligonucleotides are exposed to the cell and for a time period sufficient for the second, third and/or fourth or more labeling oligonucleotides to covalently or non-covalently bind to the prior exposed labeling oligonucleotide;

(c) ligating the one or plurality of labeling oligonucleotides to the first anchor-lipid modified oligonucleotides and, in the case of the second, third, fourth or more labeling oligonucleotides ligating the labeling oligonucleotides simultaneously to one another; and, optionally

(d) detecting the presence of the first, second, third, fourth and/or more labeling oligonucleotides by detection of one of more unique nucleotide sequences corresponding to each of the first, second, third, fourth labeling oligonucleotides; and/or

(e) isolating the cell based upon the presence of the first, second, third, fourth and/or more labeling oligonucleotides or on the sequential combination of unique nucleotide sequences of each of the first, second, third, fourth and/or more labeling oligonucleotides, wherein the presence of the first, second, third, fourth and/or more labeling oligonucleotides is determined by detection of one of more unique nucleotide sequences corresponding to the one or a combination of each of the labeling oligonucleotides.

In some embodiments, the disclosure relates to a method of identifying the spatial location of expression of endogenous nucleic acid in a cell sample, the method of isolating endogenous DNA from a cell sample, and/or the method of sequencing endogenously expressed nucleic acid sequences from a cell sample comprising:

(a) exposing a cell to one or a plurality of anchor-lipid modified oligonucleotides for a time period sufficient for the anchor-lipid modified oligonucleotide to embed itself within a cell membrane of the cell;

(b) exposing the cell to a first, second, third fourth or more labeling oligonucleotides, wherein the first labeling oligonucleotide is complementary to the one or a plurality of anchor-lipid modified oligonucleotides and exposed to the one or a plurality of anchor-lipid modified oligonucleotides for a time period sufficient for the anchor-lipid modified oligonucleotide to form a complementary strand of nucleic acid with the first labeling oligonucleotide, and wherein the second, third and/or fourth or more labeling oligonucleotides is exposed sequentially to a 3′ portion of the labeling oligonucleotide exposed to the cell immediately prior to the second, third and/or fourth or more labeling oligonucleotides are exposed to the cell and for a time period sufficient for the second, third and/or fourth or more labeling oligonucleotides to covalently or non-covalently bind to the prior exposed labeling oligonucleotide;

(c) ligating the one or plurality of labeling oligonucleotides to the first anchor-lipid modified oligonucleotides and, in the case of the second, third, fourth or more labeling oligonucleotides ligating the labeling oligonucleotides simultaneously to one another; and, optionally

(d) detecting the presence of the first, second, third, fourth and/or more labeling oligonucleotides by detection of one of more unique nucleotide sequences corresponding to each of the first, second, third, fourth labeling oligonucleotides; and/or

(e) analyzing regional expression of the cell based upon the spatial location of the first, second, third, fourth and/or more labeling oligonucleotides, or on the sequential combination of unique nucleotide sequences of each of the first, second, third, fourth and/or more labeling oligonucleotides, wherein the spatial location of the cell affected by the endogenous expression of nucleic acids is determined by detecting the spatial location of the first, second, third, fourth and/or more labeling oligonucleotides corresponding to the one or a combination of each of the labeling oligonucleotides.

In some embodiments, the cell or cell sample used in the disclosed method of labeling a cell sample, the method of isolating endogenous DNA from a cell sample, or the method of sequencing nucleic acid sequences from a cell sample is obtained by partitioning the cells corresponding to a region of the sample into a vessel. In some embodiments, the partition of the cells is performed by placing a slice or three-dimensional preparation of a tissue sample on top of the vessel and positioning the cells corresponding to a region of the sample into the vessel. In some embodiments, the vessel is a well. In some embodiments, the vessel is a microwell.

In some embodiments, the disclosure provides a method of identifying a spatial position of a pattern of nucleic acid expression within a sample or tissue of a subject, the method comprising:

(a) partitioning one or a plurality of cells from the sample or the tissue corresponding to a region of the sample into one of a plurality of vessels by placing a slice or three-dimensional preparation of the sample into one of a plurality of vessels;

(b) exposing the one or plurality of cells corresponding to a region of the sample with an known oligonucleotide disclosed herein for a time sufficient for incorporation of the known oligonucleotide into the one or plurality of cells, each oligonucleotide unique for and corresponding to one of the plurality of vessels into which one or plurality of cells are exposed;

(c) isolating and/or sequencing nucleic acids from the one or a plurality of cells according to the known oligonucleotide; and

(d) correlating the expression profile from the one or plurality of cells to the spatial position of the cells within the sample, relative to the tissue, or within the tissue.

In such embodiments, the known oligonucleotide in each of the plurality of vessels is independently selected from one or a combination of:

(x) a first lipid-conjugated DNA oligonucleotide comprising a first lipid moiety, a first hybridization region, and a first primer region; and/or

(y) a second lipid-conjugated DNA oligonucleotide comprising a second hybridization region and a second lipid moiety, wherein the second hybridization region is the reverse complement of the first hybridization region; and/or

(z) a third DNA oligonucleotide comprising from a second primer region, a barcode region, and a capture sequence, wherein the second primer region is the reverse complement of the first primer region.

In some embodiments, the disclosure provides a method of identifying a spatial expression pattern of a nucleic acid within a tissue of a subject, the method comprising:

(a) partitioning one or a plurality of cells from a sample corresponding to a region of the tissue into one of a plurality of vessels;

(b) exposing the one or plurality of cells with a lipid-conjugated DNA oligonucleotide comprising a lipid moiety as defined elsewhere herein, a barcode region as defined elsewhere herein, and a capture sequence as defined elsewhere herein for a time period sufficient for the lipid moiety to embed itself within cell membrane of the one or plurality of cells, wherein the barcode region of the lipid-conjugated DNA oligonucleotide is unique for each of the one of plurality of vessels in which the one or plurality of cells are exposed;

(c) sequencing nucleic acids captured by the capture sequence of the lipid-conjugated DNA oligonucleotide in the one or plurality of cells; and

(d) correlating the sequenced nucleic acids from the one or plurality of cells to the spatial position of the one or plurality of cells within the tissue and/or relative to the tissue according to the barcode region contained in each of the sequenced nucleic acids.

In some embodiments, the lipid-conjugated DNA oligonucleotide used in such methods comprises: (i) a first lipid-conjugated DNA oligonucleotide comprising the lipid moiety and a first primer region as defined elsewhere herein; and (ii) a second DNA oligonucleotide comprising a second primer region as defined elsewhere herein, the barcode region, and the capture sequence, wherein the second primer region is the reverse complement of the first primer region. In some embodiments, the first lipid-conjugated DNA oligonucleotide further comprises a first hybridization region as defined elsewhere herein. In some embodiments, such methods further comprise exposing the one or plurality of cells to a second lipid-conjugated DNA oligonucleotide before the step of sequencing, wherein the second lipid-conjugated DNA oligonucleotide comprises a second hybridization region as defined elsewhere herein and a second lipid moiety as defined elsewhere herein, wherein the second hybridization region is the reverse complement of the first hybridization region.

In some embodiments, the cell partition is performed by placing a tissue slice or a three-dimensional preparation of the tissue having an appropriate thickness as described elsewhere herein into the one of plurality of vessels. In some embodiments, the cell partition is performed by placing a tissue slice or a three-dimensional preparation of the tissue on top of the one of plurality of vessels and forcing a cell or cells corresponding to a defined region of the tissue sample into one of the one of plurality of vessels. In some embodiments, the one of plurality of vessels are microwells. Microwell arrays can be directly created using soft lithography into substrates such as SU-8. Lithography fabricated SU-8 molds can also be used to make microwells in PDMS. PDMS stamps can also be used as molds for other polymers such as TPE, NOA81, and PUMA. Alternatively, 3D printing methods such as Nanoscribe can be used to fabricate arrays. Any other options known in the art for creating microwell arrays, including but not limited to, injection molding or hot embossing, can also be used.

Depending on the size of the microwells, the spatial resolution of wells used in the disclosed methods can be about 100, 90, 80, 70, 60, 50, 40, 30, 20 or 10 microns. In some embodiments, the spatial resolution of wells is less than about 10 microns. In some embodiments, the spatial resolution of wells is about 10 microns. In some embodiments, the spatial resolution of wells is about 20 microns. In some embodiments, the spatial resolution of wells is about 30 microns. In some embodiments, the spatial resolution of wells is about 40 microns. In some embodiments, the spatial resolution of wells is about 50 microns. In some embodiments, the spatial resolution of wells is about 60 microns. In some embodiments, the spatial resolution of wells is about 70 microns. In some embodiments, the spatial resolution of wells is about 80 microns. In some embodiments, the spatial resolution of wells is about 90 microns. In some embodiments, the spatial resolution of wells is about 100 microns. In some embodiments, the spatial resolution of wells is more than about 100 microns.

In some embodiments, detection and/or analysis of the barcode oligonucleotides is performed by detection or quantification of one or a plurality of probes or labeled oligonucleotides. For instance, equipment such as the Biorad ddSEQ reader can be used to detect and analyze single cell or multiple cell samples in a multiplexed format. Nature Biotechnology volume 37, pages 916-924(2019), for instance, discloases a known method for quantification, detection and analysis of the presence or absence of barcoded samples. Such techniques are incorporated by reference in their entireties.

In some embodiments, the cell sample is a three-dimensional preparation of cells from a tissue sample of a subject. In some embodiments, the cell sample is a tissue slice. In some embodiments, the cell sample has a thickness from about 0.05 microns to about 150 microns. In some embodiments, the cell sample has a thickness from about 0.1 microns to about 99 microns. In some embodiments, the cell sample has a thickness from about 0.5 microns to about 80 microns. In some embodiments, the cell sample has a thickness from about 1 microns to about 50 microns. In some embodiments, the cell sample has a thickness from about 2 microns to about 40 microns. In some embodiments, the cell sample has a thickness from about 3 microns to about 30 microns. In some embodiments, the cell sample has a thickness from about 4 microns to about 20 microns. In some embodiments, the cell sample has a thickness of about 0.05 microns. In some embodiments, the cell sample has a thickness of about 0.1 microns. In some embodiments, the cell sample has a thickness of about 0.5 microns. In some embodiments, the cell sample has a thickness of about 1 microns. In some embodiments, the cell sample has a thickness of about 5 microns. In some embodiments, the cell sample has a thickness of about 10 microns. In some embodiments, the cell sample has a thickness of about 20 microns. In some embodiments, the cell sample has a thickness of about 30 microns. In some embodiments, the cell sample has a thickness of about 40 microns. In some embodiments, the cell sample has a thickness of about 50 microns. In some embodiments, the cell sample has a thickness of about 60 microns. In some embodiments, the cell sample has a thickness of about 70 microns. In some embodiments, the cell sample has a thickness of about 80 microns. In some embodiments, the cell sample has a thickness of about 90 microns. In some embodiments, the cell sample has a thickness of about 100 microns. In some embodiments, the cell sample has a thickness of about 150 microns. In some embodiments, the cell sample has a thickness of more than about 150 microns.

In some embodiments, the disclosure relates to a method of labeling a plurality of cells from a sample, and, if the method comprises exposing the cell to a plurality of labeling oligonucleotides, the method further comprises a step of pooling the cells in a single vessel before exposing the cell to each sequential step of exposure to a second, third, fourth or more labeling oligonucleotide. In some embodiments, the method further comprises a step of pooling the cells in a single vessel after exposing the cell to each sequential step of exposure to a second, third, fourth or more labeling oligonucleotide.

In one aspect, microfluidic droplets are used, for example, to contain cells. Microfluidic droplets may be used to keep the cells of a plurality of cells separate and identifiable, e.g., such that differences between the different cells may be identified. A plurality of cells, some or all of which may contain individual differences, may be studied, at resolutions down to the single-cell level, for example, by using the lipid-modified or hydrophobic-anchored oligonucleotides disclosed herein.

The cell samples may arise from any subject, e.g., a human, or from a non-human animal, for example, an invertebrate cell (e.g., a cell from a fruit fly), a fish cell (e.g., a zebrafish cell), an amphibian cell (e.g., a frog cell), a reptile cell, a bird cell, or a mammal cell, such as a monkey, ape, cow, sheep, goat, horse, donkey, camel, llama, alpaca, rabbit, pig, mouse, rat, guinea pig, hamster, dog, cat, etc. If the cell sample is from a multicellular organism, the cell sample may be from any part of the organism. In some embodiments, a tissue may be studied. For example, a tissue from an organism may be processed to produce cell samples (e.g., through slicing the tissue), such that the differences within the tissue may be determined, as discussed herein.

The cell samples or tissues may arise from a healthy subject, or one that is diseased or suspected of being diseased. For example, cell samples or tissues from a subject may be removed and studied to determine differences or changes in the profile of those cell samples or tissues, e.g., to determine if the subject is healthy or has a disease, for example, if the animal has cancer (e.g., by determining cancer specific expression profile within the cell samples or tissues). In some cases, a tumor may be studied (e.g., using a biopsy), and the profile of the tumor may be determined.

The droplets may be contained in a microfluidic channel. For example, in certain embodiments, the droplets may have an average dimension or diameter of less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 10 micrometers, less than about 5 micrometers, less than about 3 micrometers, or less than about 1 micrometer in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain instances. The droplets may be spherical or non-spherical. The average diameter or dimension of a droplet, if the droplet is non-spherical, may be taken as the diameter of a perfect sphere having the same volume as the non-spherical droplet.

The droplets may be produced using any suitable technique. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focus junction, or any other suitable junction for creating droplets. See, for example, WO 2004/091763 and WO 2004/002627, each of which is incorporated herein by reference in its entirety. In some embodiments, the junction may be configured and arranged to produce substantially monodisperse droplets.

In some cases, the cells may be encapsulated within the droplets at a relatively high rate. For example, the rate of cell encapsulation in droplets may be at least about 10 cells/s, at least about 30 cells/s, at least about 100 cells/s, at least about 300 cells/s, at least about 1,000 cells/s, at least about 3,000 cells/s, at least about 10,000 cells/s, at least about 30,000 cells/s, at least about 100,000 cells/s, at least about 300,000 cells/s, or at least about 10⁶ cells/s.

PCR reactions (including, for example, reverse transcription PCR and primer extension PCR utilizing the lipid-modified or hydrophobic-anchored oligonucleotides disclosed herein) can be performed using, e.g., any microfluidic device (including, e.g., microfluidic devices interfaced with a multisample nanodispenser). Microfluidic devices are fluid systems in which the volumes of fluid are small, typically on the order of microliters to nanoliters. In some embodiments the microfluidics can handle tens to thousands of samples in small volumes. Microfluidics can be active or passive. By using active elements such as valves in the microfluidic device, microfluidic circuits can be created. This allows not only the use of small reagent volumes but also a high task parallelization since several procedures can be processed and physically be fitted on the same chip.

In microfluidic channels, the flow of liquid can be completely laminar, that is, all of the fluid moves in the same direction and at the same speed. Unlike turbulent flow, this allows the transport of molecules in the fluid to be very predictable. Microfluidic devices can be made of glass or plastic. In some embodiments, polydimethylsiloxane (PDMS), a type of silicone can be used. Some advantages of PDMS include inexpensive, optically clear and permeable to several substances, including gases. In some embodiments, soft lithography or micromolding can be used to create PDMS based microfluidic devices. The devices can use pressure driven flow, electrodynamic flow, or wetting driven flow.

In some embodiments, the microfluidic device has multiple chambers, each chamber having a real-time microarray. In some embodiments, the array is incorporated into the microfluidic device. In some embodiments, the microfluidic device is formed by adding features to a planar surface having multiple real-time microarrays in order to create chambers wherein the chamber correspond to the real-time microarray. In some embodiments, a substrate having 3-dimensional features, for example a PDMS surface with wells, microwells, or channels, is placed in contact with a surface having multiple real-time microarrays to form a microfluidic device having multiple arrays in multiple chambers.

A device having multiple chambers, each with a real-time microarray can be used in order to analyze multiple samples simultaneously. In some embodiments, multiple chambers have sample fluid derived from the same sample. Having sample fluid from the same sample in multiple chambers can be useful, for example to measure each with different arrays for analyzing different aspects of the same sample, or for example for increasing accuracy by parallel measurements on identical arrays. In some cases, different amplicons within the same sample will have different optimum temperature profile conditions. Thus, in some embodiments, the same sample is divided into different fluid volumes, and the different fluid volumes are in different chambers with real-time microarrays; and at least some of the different fluid volumes are given a different temperature cycle.

In some embodiments, multiple chambers have sample fluid from different sources. Having sample fluid from different sources can be useful in order to increase throughput by measuring more samples in a given time period on a given instrument. In some embodiments, the microfluidic with multiple chambers containing real-time microarrays can be used for diagnostic applications. The device may have about 2, 3, 4, 5, 6, 7, 8, 9, 10, 10-15, 15-20, 20-30, 30-50, 50-75, 75-100, or more than 100 chambers each having a real time microarray.

Some embodiments relate to use of the lipid-modified or hydrophobic-anchored oligonucleotides in conjunction with solid supports. “Solid support” refers to any substrate having a surface to which molecules may be attached, directly or indirectly, through either covalent or non-covalent bonds. The solid support may include any substrate material that is capable of providing physical support for the probes that are attached to the surface. The material is generally capable of enduring conditions related to the attachment of the barcode oligonucleotides to the surface and any subsequent treatment, handling, or processing encountered during the performance of an assay. The materials may be naturally occurring, synthetic, or a modification of a naturally occurring material. Suitable solid support materials may include silicon, graphite, mirrored surfaces, laminates, ceramics, plastics (including polymers such as, e.g., poly(vinyl chloride), cyclo-olefin copolymers, polyacrylamide, polyacrylate, polyethylene, polypropylene, poly(4-methylbutene), polystyrene, polymethacrylate, poly(ethylene terephthalate), polytetrafluoroethylene (PTFE or Teflon®), nylon, poly(vinyl butyrate)), germanium, gallium arsenide, gold, silver, etc., either used by themselves or in conjunction with other materials. Additional rigid materials may be considered, such as glass, which includes silica and further includes, for example, glass that is available as Bioglass. Other materials that may be employed include porous materials, such as, for example, controlled pore glass beads. Any other materials known in the art that are capable of having one or more functional groups, such as any of an amino, carboxyl, thiol, or hydroxyl functional group, for example, incorporated on its surface, are also contemplated.

The material used for a solid support may take any of a variety of configurations ranging from simple to complex. The solid support can have any one of a number of shapes, including a strip, plate, disk, rod, particle, including bead, tube, well, and the like. Usually, the material is relatively planar such as, for example, a slide, though it can be spherical, such as, for example, a bead, or cylindrical (e.g., a column). In many embodiments, the material is shaped generally as a rectangular solid. Multiple predetermined arrangements such as, e.g., arrays of probes, may be synthesized on a sheet, which is then diced, i.e., cut by breaking along score lines, into single array substrates. Exemplary solid supports that may be used include microtiter wells, microscope slides, membranes, magnetic beads, charged paper, Langmuir-Blodgett films, silicon wafer chips, flow through chips, and microbeads. In some embodiments, the beads are plastic or polystyrene. In some embodiments, the beads are magnetic. After exposure of the beads to a magnetic force, nucleic acid molecules or sequences form the cells are capable of being isolated. Individual DNA and RNA

Direct conjugation of the barcode oligonucleotide via its capture sequence, such as a poly(A) tail, to the solid support is possible in some embodiments. In such embodiments, cells or membranes comprising the anchor, and optionally co-anchor, lipid-modified or hydrophobic-anchored oligonucleotides can be exposed to the solid support in hybridizing conditions such that cells or membranes comprising the anchor, and optionally co-anchor, lipid-modified or hydrophobic-anchored oligonucleotides bind to the barcode oligonucleotide. The solid support can then be washed of non-binding substances and remaining solid support including bound cells and membranes further analyzed by sequencing or other identification scheme.

In some embodiments, the solid support is unbound initially and binds to a barcode oligonucleotide through the barcode oligonucleotide's capture sequence. In such embodiments, cells or membranes comprising the anchor, and optionally co-anchor, lipid-modified or hydrophobic-anchored oligonucleotides have already hybridized to the barcode oligonucleotide through the first and second primer regions. The solid support can then be washed of non-binding substances and remaining solid support including bound cells and membranes further analyzed by sequencing or other identification scheme.

In some embodiments, the disclosure relates to a method of labeling a cell with a barcode or lipid-modified oligonucleotide disclosed herein. In some embodiments, the methods comprise contacting one or a plurality of homogenous mixture or heterogenous mixture of the disclosed oligonucleotides with one or a plurality of cells from a sample or tissue. In some embodiments, the cells are contacted with at least a first and a second lipid-modified oligonucleotide, such that the lipid-oligonucleotide hybridizes with an RNA, DNA, or RNA/DNA hybrid present in the cell. In some embodiments, the capture region of the oligonucleotide is used to isolate the oligonucleotide to a solid support or another fixed oligonucleotide such that unhybridized elements or components from the cells may be removed and a retentate of the captured RNA/DNA from the cell is preserved. In some embodiments, the captured RNA/DNA from a single cell may be isolated in a culture vessel. In some embodiments, a plurality of captured DNA/RNA sequences may be maintained in a library corresponding to the cell from which it was isolated. In some embodiments, the plurality of captured DNA/RNA sequences are sequenced, such that the sequenced DNA or RNA corresponds to an expression pattern of that cell.

The disclosure relates to a method of preparing a library of oligonucleotides expressed by a single cell or multiple cells in isolation, the method of generating the library comprising sequencing RNA or DNA from the one or multiple cells after the one or multiple cells are exposed to one or a plurality of lipid-modified oligonucleotides disclosed herein. In some embodiments, endogenous nucleotides from one or a plurality of cells can be isolated and/or identified by correlating a known signal or frequency of a probe bound to the lipid-modified oligonucleotide to the cell upon which the oligonucleotide was bound. The signal or frequency of the probe can be paired with the source of the endogenous DNA and/or RNA. In some embodiments, the endogenous nucleotides are expressed mRNA from the one or plurality of cells or cDNA that has been formed from constructing a library of complementary strand of mRNA by, for example, PCR or other known technique to create cDNA library from isolated mRNA. In some embodiments, cDNA or mRNA isolated from a single cell by contacting the cell to one or plurality of lipid-modified oligonucleotide disclosed herein such that each cell captured can be correlated to the identify, number or detection of a probe bound to the lipid-modified oligonucleotide or oligonucleotides. Different distributions of one or a plurality of lipid-modified oligonucleotides on one or plurality of cells can be used to correlate that cell with its particular set of endogenous expression patterns. Cells carrying one or two or more lipid-modified oligonucleotides may be isolated using an antibody specific for an antigen or other protein on the surface of the cells. An expression pattern of a cell (created by sequencing one or a plurality of endogenous sequences expressed by the cell) can be correlated to a corresponding identity of a cell with a known antigen identified by adhesion of an antibody known to bind the target sequence of the antibody.

### EXAMPLES

### General Spatial Information

LMOs can also be used to barcode cells based on the relative spatial orientation. There are, generally speaking, two approaches to achieving spatial barcoding: (1) physical separation cells/tissue regions followed by barcoding as described previously and; (2) addition of lipid-modified oligonucleotide anchors to cells followed by addition of spatially-defined barcode oligonucleotides. In first case, the physical separation can be achieved across a range of length scales through dissection with scalpel, microwell isolation, or laser-capture microdissection. Once cells are isolated, barcodes can be introduced to each unique sample indicating the relative location of the cells in that sample. In the second case, all cells receive anchor and co-anchor before addition of barcode oligonucleotides. The barcodes are specific to a location and diffuse from the location of introduction to be captured on cells via hybridization to the anchor strand. The relative locations of cells are determined by the amount and relative ratios of the spatial barcodes. Introduction of spatial barcodes can be achieved by several methods including microarrayers, inkjet printers, acoustic liquid handlers, and cleavage from a solid-support (e.g., array or beads).

### Spatial Barcoding of the Developing Gut (PROPHETIC)

To apply MULTI-seq for mesoscale spatial barcoding of scRNA-seq samples, we first will surgically-removed the small intestine from freshly-euthanized mice and dissected or from dissected embryos. The small intestine will be then stretched along a surface before connective tissue and fat is removed with a scalpel. The small intestine will be then filleted prior to 4× washes with ice-cold PBS with shaking. After washing, each intestine will be cut into equally-sized segments (i.e., from about 1 to about 100 microns in length along the proximal-distal axis for adult intestines; from about 1 to about 100 microns for developing intestine). Segments will then be dissociated for 20 minutes at room temperature with shaking in 2 mL dissociation media (RPMI1640 with 3% FBS, 1% pen/strep, 1% sodium pyruvate, 1% MEM non-essential amino acids, 1% L-glutamine, 2.5% HEPES, 5 mM EDTA and 10 mM DTT).

Following dissociation and manual agitation with a p1000 pipette, the dissociation solution will be strained through a 100 mM filter into 15 mL conical vial on ice. Tissue chunks remaining at the top of strainer will then be transferred to a separate 15 mL conical vial containing 4 mL EDTA for further digestion (e.g., vigorous shaking for 30 seconds) prior to re-filtering. This process will be repeated twice, generating a roughly-filtered cell suspension. This roughly-filtered suspension will be then filtered through a 70 mM filter into a new 15 mL conical vial prior to centrifugation for 8 minutes at 1500 rpm.

Each cell suspension will then be washed once with 10 mL ice-cold PBS prior to resuspension to a single-cell suspension in 160 mL in ice-cold PBS. Single-cell suspensions will be then transferred to individual wells of a 48-well plate before adding 20 mL of 2.5 mM anchor LMO pre-hybridized to a single MULTI-seq sample barcode. Cell suspensions will then manually agitated and incubated on ice for 5 minutes prior to addition of 20 mL of 2.5 mM co-anchor LMO and a subsequent 5 minute incubation on ice. Following MULTI-seq labeling, labeling solutions will be diluted with 300 mL with 5% BSA to quench ambient LMOs prior to pooling, antibody staining, and FACS enrichment for live cells. Live cells will be then subjected to the standard 10× Genomics scRNA-seq workflow using droplet microfluidics.

### Physical Separation Using Microwells

Microwell arrays can be directly created using soft lithography into substrates such as SU-8. Lithography fabricated SU-8 molds can also be used to make microwells in PDMS. Furthermore, PDMS stamps can be used as molds for other polymers such as TPE, NOA81, and PUMA. Alternatively 3D printing methods such as Nanoscribe can be used to fabricate arrays. Other options to create arrays are to use injection molding or hot embossing. The spatial resolution of wells can be 100, 90, 80, 70, 60, 50, 40, 30, 20 or 10 microns.

Once fabricated, tissue dissociation reagents can be deposited into wells. Additionally, 20 to 500 nM anchor with different and defined barcodes or combination of barcodes will be added to each well, providing spatial information. A tissue slice is placed on the array and clamped down with a seal. This forces tissue into the wells. After dissociation, the seal is removed, and the array is placed in a solution containing a quenching reagent and a non-quenched co-anchor. Cells are washed and filtered through a cell strainer and sorted for intact cells.

Cells can be further processed using any number of commercial single cell sequencing platforms such as 10× Genomics, Bio-Rad ddSEQ, CelSee Genesis, etc. The MULTI-seq barcodes or barcode combinations associated with each cell reveal the spatial location of that cell in the original array and can be tied back to the transcriptional information from that cell.

### Diffusion-Based Method Using Oligonucleotide Barcode Array

A tissue slice is placed on a microarray with oligonucleotide barcodes at <10 μM resolution. Anchor (100 nM to 2 μM) is washed over the tissue slice and allowed to diffuse through the tissue before addition of equimolar CoAnchor. The spatial barcodes are then released (e.g., cleavage of disulfide linker) and allowed to diffuse through the tissue. The tissue is then dissociated and can be further processed using any number of commercial single cell sequencing platforms such as 10× Genomics, Bio-Rad ddSEQ, CelSee Genesis, etc. Cell location information can be determined by the relative amount of each spatial barcode.

### Alternative Ways to Introduce Barcodes for Diffusion-Based Method

Instead of using an oligonucleotide array, barcoding reagents can be delivered to tissue directly a solution containing tissue with an array of printing pins, using an acoustic liquid handler such as the Labcyte Echo or EDC ATS. Barcoding reagents can also be delivered to tissue into a microfluidic device by flowing different barcodes to different regions of tissue along different axes.

1. A composition comprising:
(a) a first lipid-conjugated DNA oligonucleotide comprising a first lipid moiety, a first hybridization region, and a first primer region;
(b) a second lipid-conjugated DNA oligonucleotide comprising a second hybridization region and a second lipid moiety, wherein the second hybridization region is the reverse complement of the first hybridization region; and
(c) a third DNA oligonucleotide comprising a second primer region, a barcode region, and a capture sequence, wherein the second primer region is the reverse complement of the first primer region.

2. The composition of claim 1, wherein the first lipid-conjugated DNA oligonucleotide comprises the first lipid moiety, the first hybridization region, and the first primer region.

3. (canceled)

4. The composition of claim 1, wherein the second lipid-conjugated DNA oligonucleotide comprises the second hybridization region and the second lipid moiety.

5. (canceled)

6. The composition of claim 1, wherein the third DNA oligonucleotide comprises the second primer region, the barcode region, and the capture sequence.

7.-8. (canceled)

9. The composition of claim 1, wherein the first lipid moiety comprises a compound of Formula I: 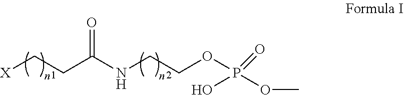
or a physiologically acceptable salt thereof,
wherein n¹ is from 5 to 25, n² is from 1 to 25, and X is selected from the group consisting of NH, CH₂, O, and CH—R, wherein R is a C12 to C28 monoglyceride, alkenyl, alkyl, aryl, or aralkyl.

10. The composition of claim 1, wherein the second lipid moiety comprises a compound of Formula II: 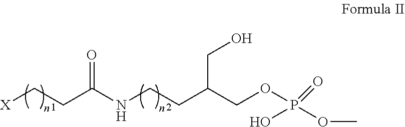
or a physiologically acceptable salt thereof,
wherein n¹ is from 5 to 25, n² is from about 0 to about 24, and X is selected from the group consisting of NH, CH₂, O, and CH—R, wherein R is a C12 to C28 monoglyceride, alkenyl, alkyl, aryl, or aralkyl.

11. The composition of claim 1, wherein the first lipid moiety comprises a lipid selected from lignoceric acid and cholesterol.

12. The composition of claim 1, wherein the second lipid moiety comprises a lipid selected from palmitic acid and cholesterol.

13.-17. (canceled)

18. The composition of claim 1, wherein one or more of the first lipid-conjugated DNA oligonucleotide, the second lipid-conjugated DNA oligonucleotide, and the third lipid-conjugated DNA oligonucleotide is bound to a solid support.

19. The composition of claim 18, wherein the solid support is a bead; and wherein the capture sequence comprises a polyadenylation region and the bead comprises a poly(T) region that hybridizes to the polyadenylation region of the third DNA oligonucleotide.

20.-22. (canceled)

23. A method of labeling a cell sample, the method comprising:
(a) exposing the cell sample to one or a plurality of anchor-lipid modified oligonucleotides disclosed herein or a composition of claim 1 for a time period sufficient for the anchor-lipid modified oligonucleotide to embed itself within a cell membrane of the cell;
(b) exposing the cell sample to one or a plurality of labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides;
(c) ligating the one or plurality of labeling oligonucleotides to the one or plurality of anchor-lipid modified oligonucleotides; and, optionally
(d) detecting the presence of the one or plurality of labeling oligonucleotides by detection of one of more unique nucleotide sequences corresponding to the one or plurality of labeling oligonucleotides; and/or
(e) isolating the cell based upon the presence of one or plurality of labeling oligonucleotides, wherein the presence of the one or plurality of labeling oligonucleotides is determined by detection of one of more unique nucleotide sequences corresponding to the one or plurality of labeling oligonucleotides.

24.-25. (canceled)

26. The method of claim 23, further comprising a step of:
(f) sequencing the nucleic acid from the one or plurality of cells.

27. (canceled)

28. The method of claim 23, further comprising a step of creating an expression profile of each of the one or plurality of cells corresponding to a region of the sample; and a step of correlating the sequence information and/or the expression profile of the nucleic acid to a spatial position of the one or plurality of cells within the sample or within the subject.

29. (canceled)

30. The method of claim 23, wherein the step of labeling comprises:
(w) exposing the one or plurality of cells to one or a plurality of anchor-lipid modified oligonucleotides for a time period sufficient for the anchor-lipid modified oligonucleotide to embed itself within a cell membrane of the cell; and/or
(x) exposing the one or plurality of cells to one or a plurality of labeling oligonucleotides complementary to the anchor-lipid modified oligonucleotides for a time period sufficient for the anchor-lipid modified oligonucleotide to form a complementary strand of nucleic acid with the one or plurality of labeling oligonucleotides; and/or
(y) ligating the one or plurality of labeling oligonucleotides to the one or plurality of anchor-lipid modified oligonucleotides; and, optionally
(z) detecting the presence of the one or plurality of labeling oligonucleotides by detection of one or more unique nucleotide sequences corresponding to the one or plurality of labeling oligonucleotides.

31. The method of claim 30, wherein the step of isolating the cell comprises isolating the cell based upon the presence of one or plurality of labeling oligonucleotides, wherein the presence of the one or plurality of labeling oligonucleotides is determined by detection of one of more unique nucleotide sequences corresponding to the one or plurality of labeling oligonucleotides.

32.-51. (canceled)

52. A method of identifying a spatial position of a pattern of nucleic acid expression within a sample or tissue of a subject, the method comprising:
(a) partitioning one or a plurality of cells from the sample or the tissue corresponding to a region of the sample into one of a plurality of vessels;
(b) exposing the one or plurality of cells corresponding to a region of the sample with an known oligonucleotide disclosed herein for a time sufficient for incorporation of the known oligonucleotide into the one or plurality of cells, each oligonucleotide unique for and corresponding to one of the plurality of vessels into which one or plurality of cells are exposed;
(c) isolating nucleic acid from the one or a plurality of cells according to the known oligonucleotide;
(d) quantifying expression of nucleic acids and/or sequencing the nucleic acid from the one or a plurality of cells;
(e) normalizing the expression of nucleic acid in an expression profile; and
(f) correlating the expression profile from the one or plurality of cells to the spatial position of the cells within the sample, relative to the tissue, or within the tissue,
wherein the known oligonucleotide disclosed herein in each of the plurality of vessels is independently selected from one or a combination of:
(x) a first lipid-conjugated DNA oligonucleotide comprising a first lipid moiety, a first hybridization region, and a first primer region; and/or
(y) a second lipid-conjugated DNA oligonucleotide comprising a second hybridization region and a second lipid moiety, wherein the second hybridization region is the reverse complement of the first hybridization region; and/or
(z) a third DNA oligonucleotide comprising from a second primer region, a barcode region, and a capture sequence, wherein the second primer region is the reverse complement of the first primer region;
wherein the step of partitioning comprises placing a slice or three-dimensional preparation of the sample from about 0.1 to about 99 microns in thickness into one of a plurality of vessels.

53.-77. (canceled)

78. A method of identifying a spatial expression pattern of a nucleic acid within a tissue of a subject, the method comprising:
(a) partitioning one or a plurality of cells from a sample corresponding to a region of the tissue into one of a plurality of vessels;
(b) exposing the one or plurality of cells with a lipid-conjugated DNA oligonucleotide comprising a lipid moiety, a barcode region, and a capture sequence for a time period sufficient for the lipid moiety to embed itself within cell membrane of the one or plurality of cells, wherein the barcode region of the lipid-conjugated DNA oligonucleotide is unique for each of the one of plurality of vessels in which the one or plurality of cells are exposed;
(c) sequencing nucleic acids captured by the capture sequence of the lipid-conjugated DNA oligonucleotide in the one or plurality of cells; and
(d) correlating the sequenced nucleic acids from the one or plurality of cells to the spatial position of the one or plurality of cells within the tissue and/or relative to the tissue according to the barcode region contained in each of the sequenced nucleic acids.

79. The method of claim 78, wherein the lipid-conjugated DNA oligonucleotide comprises:
(i) a first lipid-conjugated DNA oligonucleotide comprising the lipid moiety and a first primer region; and
(ii) a second DNA oligonucleotide comprising a second primer region, the barcode region, and the capture sequence, wherein the second primer region is the reverse complement of the first primer region.

80. The method of claim 79, wherein the first lipid-conjugated DNA oligonucleotide further comprises a first hybridization region.

81-82. (canceled)

83. The method of claim 78, wherein the first lipid moiety comprises a compound of Formula I: 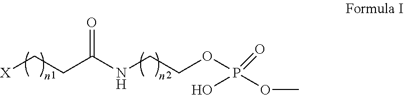
or a physiologically acceptable salt thereof,
wherein n¹ is from 5 to 25, n² is from 1 to 25, and X is selected from the group consisting of NH, CH₂, O, and CH—R, wherein R is a C12 to C28 monoglyceride, alkenyl, alkyl, aryl, or aralkyl.

84. The method of claim 78, wherein the second lipid moiety comprises a compound of Formula II: 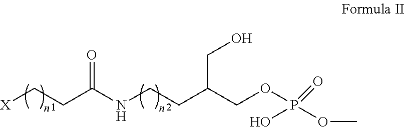
or a physiologically acceptable salt thereof,
wherein n¹ is from 5 to 25, n² is from 0 to 24, and X is selected from the group consisting of NH, CH₂, O, and CH—R, wherein R is a C12 to C28 monoglyceride, alkenyl, alkyl, aryl, or aralkyl.

85.-95. (canceled)
